(19) United States
(12) Patent Application Publication
(10) Pub. No.: US 2006/0110390 A1
(43) Pub. Date: May 25, 2006

(54) Inhibition of Ku as a treatment for cardiovascular diseases

(75) Inventors: Leslie Leinwand, Boulder, CO (US); Carmen Sucharov, Superior, CO (US)

Correspondence Address: FULBRIGHT & JAWORSKI L.L.P., 600 CONGRESS AVE., SUITE 2400, AUSTIN, TX, 78701, US

(73) Assignee: Myogen, Inc.

(21) Appl. No.: 11/203,828

(22) Filed: Aug. 15, 2005

Related U.S. Application Data
(60) Provisional application No. 60/604,435, filed on Aug. 25, 2004.

(51) Int. Cl. A61K 48/00; A61K 38/54; A61K 39/395

(52) U.S. Cl. 424/146.1; 514/44; 514/2

(57) ABSTRACT

The present invention provides for methods of treating and preventing cardiovascular diseases, in particular pathological cardiac hypertrophy and chronic heart failure, by applying an inhibitor of Ku. The present invention also provides for methods of screening to find inhibitors of Ku and inhibitors of cardiac hypertrophy and heart failure.

This application claims benefit of priority to U.S. Provisional Application Ser. No. 60/604,435 filed Aug. 24, 2004, the entire contents of which are hereby incorporated by reference.

The government owns rights in the invention pursuant to funding from the National Institutes of Health (NIH RO1 HL56510).

### BACKGROUND OF THE INVENTION

1. Field of the Invention

The present invention relates generally to the fields of developmental biology and molecular biology. More particularly, it concerns gene regulation and cellular physiology in cardiomyocytes. Specifically, the invention relates to the use of inhibitors of Ku protein to treat cardiac hypertrophy and heart failure, and to screening methods for finding inhibitors of Ku as well as inhibitors of heart failure and cardiac hypertrophy.

2. Description of Related Art

Cardiovascular diseases encompass a wide variety of etiologies and have an equally wide variety of causative agents and interrelated players. Heart failure, for example, is a pathophysiological state in which the heart fails to pump blood at a rate commensurate with the requirements of the metabolizing tissues of the body. Heart failure is caused in most cases—about 95% of the time—by myocardial failure. Cardiac hypertrophy, a form of heart failure as well as a cardiovascular disease in and of itself, is an adaptive response of the heart to many forms of stress or insult, including hypertension, mechanical load abnormalities, myocardial infarction, valvular dysfunction, certain cardiac arrhythmias, endocrine disorders and genetic mutations in cardiac contractile protein genes. While the hypertrophic response is thought to be an initially compensatory mechanism that augments cardiac performance, sustained hypertrophy is maladaptive and frequently leads to ventricular dilation and the clinical syndrome of heart failure. Accordingly, cardiac hypertrophy has been established as an independent risk factor for cardiac morbidity and mortality.

The contractile proteins of the heart lie within the muscle cells, called myocytes, which constitute about 75% of the total volume of the myocardium. The two major contractile proteins are the thin actin filament and the thick myosin filament. Each myosin filament contains two heavy chains (MyHC) and four light chains. The bodies of the heavy chains are intertwined, and each heavy chain ends in a head. Each lobe of the bi-lobed myosin head has an ATP-binding pocket, which has in close proximity the myosin ATPase activity that breaks down ATP to its products.

The velocity of cardiac muscle contraction is controlled by the degree of ATPase activity in the head regions of the myosin molecules. The major determinant of myosin ATPase activity and, therefore, of the speed of muscle contraction, is the relative amount of the two myosin heavy chain isomers, α and β (MyHC). The α-MyHC isoform has approximately 2-3 times more enzymatic activity than the β-MyHC isoform and, consequently, the velocity of cardiac muscle shortening is related to the relative percentages of each isoform. For example, adult rodent ventricular myocardium has approximately 80-90% α-MyHC, and only 10-20% β-MyHC, which explains why its myosin ATPase activity is 3-4 times greater than bovine ventricular myocardium, which contains 80-90% β-MyHC.

When ventricular myocardial hypertrophy or heart failure is created in rodent models, a change occurs in the expression of MyHC isoforms, with α-MyHC decreasing and β-MyHC increasing. These “isoform switches” reduce the contractility of the hypertrophied rodent ventricle, ultimately leading to myocardial failure. This pattern of altered MyHC gene expression has been referred to as reversion to a “fetal” expression pattern because, during fetal and early neonatal development, β-MyHC also dominates in rodent ventricular myocardium.

It has been shown that myocardial function declines with age in animals. Cellular and molecular mechanisms that account for age-associated changes in myocardial performance have been studied largely in rodents. Among other changes, marked shifts in MyHC occur in rodents, i.e., the β isoform becomes predominant in senescent rats. Steady-state mRNA levels for α-MyHC and β-MyHC parallel the age-associated change in the MyHC proteins. The myosin ATPase activity declines with the decline in α-MyHC content, and the altered cellular profile results in a contraction that exhibits a reduced velocity and a prolonged time course.

Human atrial myocardium undergoes similar isoform switches with hypertrophy or failure. In the past, several studies examined this issue in autopsy cases, but did not find biologically significant expression of the α-MyHC isoform in putatively normal hearts. Since there was thought to be no significant expression of α-MyHC in normal hearts, a down-regulation in α-MyHC was not thought to be a possible basis for myocardial failure in humans. There was one early report that the amount of α-MyHC, although extremely small to begin with, was reduced in failing human myocardium. (Bouvagnet, 1989). However, more recent reports have shown the existence of appreciable levels of α-MyHC in the human heart at both the mRNA and protein level. At the mRNA level, 23-34% of the total ventricular mRNA is derived from α-MyHC (Lowes et al., 1997; Nakao et al., 1997), while approximately 1-10% of the total myosin protein content is α-MyHC (Miyata et al., 2000; Reiser et al., 2001). These changes in MyHC isoform content are sufficent to explain the decrease in myosin or myofibrillar ATPase activity in the failing human heart (Hajjar et al., 1992; Pagani et al., 1988).

Data generated in the 1990's suggested that β myosin heavy chain mutations may account for approximately 30-40% percent of cases of familial hypertrophic cardiomyopathy (Watkins et al., 1992; Schwartz et al., 1995; Marian and Roberts, 1995; Thierfelder et al., 1994; Watkins et al., 1995). A patient with no family history of hypertrophic cardiomyopathy presented with late-onset cardiac hypertrophy of unkonwn etiology, and was shown to have a mutation in α-MyHC (Niimura et al., 2002). Two important studies have shown even more convincingly the important role of the MyHC isoforms in cardiovascular disease. Lowes et al. (2002) showed that using beta blockers to treat dilated cardiomyopathy led to increased levels of α-MyHC and decreased levels of β-MyHC that directly corresponded to improvement in disease state. In fact, the changes in α-MyHC noted in those studies was the only factor shown to correlate with improvement in cardiac function. Equally convincingly, Abraham et al. (2002) have shown that myosin heavy chain isoform changes directly contribute to disease progression in dilated cardiomyopathy. These studies show the importance and need for an agent that can alter, if not reverse, the isoform switching that occurs in the MyHC isoforms in cardiovascular disease.

### SUMMARY OF THE INVENTION

Therefore, and in accordance with the present invention, there is provided a method of treating cardiovascular diseases such as heart failure or cardiac hypertrophy comprising inhibiting the function of Ku. Inhibition may comprise inhibiting the interaction of Ku and YY1, reducing the expression of Ku, inhibiting the binding of Ku to the α-MyHC promoter, binding to or inactivating Ku, or inhibiting (directly or indirectly) the Ku-dependent repression of the α-MyHC gene.

In certain embodiments of the invention, the agent that reduces the expression of Ku is an antisense construct, an siRNA, or a ribozyme. In other embodiments, the agent that binds to or inactivates Ku may be an antibody or antibody preparation, a Ku mimetic, a peptide, a peptide aptamer, or a small molecule. The antibody may comprise a single chain antibody, a polyclonal or a monoclonal antibody.

In specific embodiments, the method of treatment further comprises targeting the delivery of the inhibitor to the heart. The delivery may be accomplished by injection of the inhibitor directly into the heart, by use of an indwelling catheter or stent, or by use of an expression vector, viral vector, or any other cardiac specific delivery vector or gene therapy approach.

In yet further embodiments of the invention, there is provided a second therapeutic agent to the patient. The second therapeutic agent may be selected from the list consisting of but not limited to beta blockers, inotropes, diuretics, ACE-1, AII antagonists, BNP, Ca(++) channel blockers, phosphodiesterase inhibitors, endothelin receptor antagonists, or HDAC inhibitors. In certain embodiments, the second therapeutic is administered at the same time as the inhibitor of Ku, and in other embodiments the second therapeutic may be administered either before or after the inhibitor of Ku.

In specific embodiments of the invention, treatment comprises improving one or more symptoms of pathologic cardiac hypertrophy. The one or more symptoms may comprise increased exercise capacity, increased cardiac ejection volume, decreased left ventricular end diastolic pressure, decreased pulmonary capillary wedge pressure, increased cardiac output, or cardiac index, lowered pulmonary artery pressures, decreased left ventricular end systolic and diastolic dimensions, decreased left and right ventricular wall stress, decreased wall tension, increased quality of life, and decreased disease related morbidity or mortality.

In another embodiment, treatment comprises improving one or more symptoms of heart failure. The one or more symptoms may comprise progressive remodeling, ventricular dilation, decreased cardiac output, impaired pump performance, arrhythmia, fibrosis, necrosis, energy starvation, hypertrophy, or apoptosis.

In another embodiment of the invention, there is provided a method of preventing pathologic hypertrophy or heart failure comprising (a) identifying a patient at risk of developing pathologic cardiac hypertrophy or heart failure; and (b) administering to said patient an inhibitor of Ku. In specific embodiments, the inhibitor of Ku is selected from the group consisting of a Ku siRNA molecule, a Ku antisense molecule, a Ku ribozyme molecule, a peptide, a small molecule, a Ku mimetic, a Ku aptamer, or a Ku-binding single-chain antibody, or expression construct that encodes a Ku-binding single-chain antibody. The inhibitor of Ku may be administered intravenously, subcutaneously, or by direct injection into cardiac tissue, or by use of an indwelling catheter or other device such as a stent, and in yet additional embodiments of the invention said administering comprises oral, transdermal, sustained release, controlled release, delayed release, suppository, or sublingual administration.

In certain specific embodiments of the invention, the patient at risk may exhibit one or more of a list of risk factors comprising long standing uncontrolled hypertension, uncorrected valvular disease, chronic angina, recent myocardial infarction, congenital predisposition to heart disease or pathological hypertrophy. In yet further embodiments, the patient at risk may be diagnosed as having a genetic predisposition to cardiac hypertrophy or a familial history of cardiac hypertrophy.

In yet another embodiment of the invention, there is provided a method of screening for inhibitors of cardiac hypertrophy or heart failure comprising the steps of (a) providing a cell having an intact α myosin heavy chain promoter operably linked to a reporter gene, and wherein said cell expresses sufficient levels of Ku70 and Ku80 to operably repress the α-MyHC promoter; (b) contacting said cell with a candidate inhibitor; and (c) monitoring said cell for an increase in expression of the reporter in the presence of said candidate inhibitor as compared to the expression of a cell in the absence of said candidate inhibitor; wherein an increase in expression of the reporter gene in the presence of the candidate inhibitor identifies said candidate as an inhibitor of heart failure or cardiac hypertrophy.

In specific embodiments, the cell is a cardiomyocyte, and in a further embodiment the cell is a primary cardiomyocyte. In yet further embodiments, the contacting is performed either in vitro or in vivo.

In yet further embodiments, it is contemplated that the candidate inhibitor is an antisense molecule, an siRNA molecule, a ribozyme, or said candidate inhibitor may be selected from a small molecule library. The candidate inhibitor may be an antibody and may further be a single chain antibody or a monoclonal antibody.

In specific embodiments of the invention, the reporter protein used may be luciferase, β-gal, or green fluorescent protein. And in yet further embodiments, the expression level may be measured using hybridization of a nucleic acid probe to a target mRNA or amplified nucleic acid product.

In certain embodiments of the invention, expression of Ku70 and Ku80 is driven from heterologous expression constructs, while in other embodiments Ku70 and Ku80 are inducibly expressed.

Embodiments discussed with respect to one embodiment or example of the invention may be employed or implemented with respect to any other embodiment of the invention.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-D—Failing human heart extracts have elevated α-MyHC DNA binding activity. (FIG. 1A) The −370/−350 region of the human α-MyHC promoter was incubated with non-failing and failing heart extracts and assayed by EMSA. (FIG. 1B) The same probe was incubated with failing heart and HeLa cell extracts. (FIG. 1C) The binding complexes were quantified and plotted on a graph. (FIG. 1D) EMSA using a probe containing a Sp1 binding site was incubated with non-failing and failing heart extracts. (FIG. 1E) The binding complexes were quantified and plotted on a graph.

FIGS. 2A-C—Purification of the Ku70/80 complex. (FIG. 2A) EMSA of the eluted fractions from the Dynabead purification (see Methods). Lane 1: 200 mM KCl elution; Lane 2: 300 mM KCl elution; Lane 3: 1M KCl elution; 4: HeLa cell nuclear extract. (FIG. 2B) Cross-linking experiments using the eluted fractions from the Dynabead purification. Lane 1: Flow through; Lane 2: 1M KCl elution; Lane 3: 200 mM KCl elution. (FIG. 2C) Denaturing polyacrylamide gel of the 1M elution fraction. Lane 1: 1M elution fraction; Lane 2: 1 M elution of the fraction containing beads only; Lane 3: Flow through. The arrows represent the 5 bands that were analyzed by mass spectrometry. 1 and 2 are Ku70 and Ku80 respectively.

FIGS. 3A-D—The Ku70/80 complex binds to the α-MyHC promoter in a specific manner. (FIG. 3A) EMSA using the wild-type probe incubated with: HeLa cells nuclear extract (lane 1); HeLa cell nuclear extract+YY1 Ab (lane 2); HeLa cell nuclear extract+Ku70 Ab (lane 3); purified Ku proteins (lane 4); purified Ku proteins+Ku70 Ab (lane 5); purified Ku proteins+YY1 Ab (lane 6). (FIG. 3B) EMSA using the wild-type probe incubated with: failing human heart extract (lanes 1 and 3); failing heart nuclear extract+Ku70 Ab (lane 2); failing heart nuclear extract+Ku80 Ab (lane 4). (FIG. 3C) EMSA using probes containing: mutations in the YY1 binding site (lane 2); Ku binding site (lane 3); Ku and YY1 binding site (lane 4) incubated with HeLa cell nuclear extract. (FIG. 3D) EMSA using the YY1 mutant probe incubated with HeLa cell nuclear extract. Increasing amounts of the Ku WT competitor (lanes 2 and 3) and mutant competitor (lanes 4 and 5).

FIGS. 4A-B—Ku recognizes and binds to the −370/−350 region of the α-MyHC promoter independently of DNA ends. (FIG. 4A) Effect of exonuclease III on linear and circular probes (see methods). (FIG. 4B) EMSA of the linear and circular probe incubated with HeLa cell nuclear extract and purified Ku70/80 proteins+/−exonuclease III. (FIG. 4C) Schematic representation of the linear and circular probes.

FIG. 5—Ku70 protein levels are increased in failing heart extracts. Western blot experiments using Ku70 Ab. Lanes 1-4: nuclear extract from non-failing human hearts; Lanes 5-8: nuclear extract from failing human hearts. The relative amount of Ku70 protein is plotted in the graph.

FIGS. 6A-C—The Ku70/80 complex represses the activity of the α-MyHC promoter in NRVMs. (FIG. 6A) Co-transfection of Ku70/80 represses the activity of the α-MyHC promoter. (FIG. 6B) Mutations in the Ku binding site result in a 5 fold up-regulation of the α-MyHC promoter. (FIG. 6C) Co-transfection of Ku70/80 does not change the activity of the ANF promoter.

FIGS. 7A-C—YY1 interacts with Ku70 and Ku80. HeLa cell extracts were incubated with antibodies indicated at the botton of each panel and Western blot experiments were performed with the antibodies as indicated. (FIG. 7A) Western blot of Ku70. (FIG. 7B) Western blot of Ku80. (FIG. 7C) Western blot of YY1.

FIGS. 8A-B—YY1 and Ku70/80 together increase the repression of α-MyHC promoter activity. (FIG. 8A) Effects of YY1 and Ku70/80 in NRVMs on the activity of the α-MyHC promoter. The results are normalized to the α-MyHC promoter co-transfected with an empty vector. (FIG. 8B) Co-transfection of Ku70/80 and an α-MyHC promoter construct containing the YY1 or the Ku binding sites mutated in NRVMs results in lower repression levels. The wild-type and mutant constructs are normalized to 100%. The activity of the mutant construct is higher than that of the wild-type (Sucharov et al., 1995 and FIG. 6).

FIGS. 9A-C—Ku70 and Ku80 repress endogenous α-MyHC gene expression. (FIG. 9A) Over-expression of Ku70 and Ku80 together but neither one alone results in increased levels of both proteins. Western blot of NRVMs infected with adenovirus expressing Ku70 and/or Ku80. Lanes 1-3: Ku70 Ab; Lanes 4-6: Ku80 Ab. Lanes 1 and 4: Nuclear extract from NRVMs infected with a control adenovirus; Lane 2: Nuclear extract from NRVMs infected with adenovirus expressing Ku70; Lanes 3 and 6: Nuclear extract from NRVMs infected with adenovirus expressing Ku70 and Ku80; Lane 5: Nuclear extract from NRVMs infected with adenovirus expressing Ku80. (FIG. 9B) Representative RPA. Lane 1: Infection with control virus; Lane 2: Infection with Ku70 and Ku80 viruses. (FIG. 9C) Endogenous αMyHC mRNA levels are repressed by over expression of Ku70 and Ku80. RPA experiments were done and the results are plotted.

FIGS. 10A-B—Neonate Rat Cardiac Myocytes were infected with an adenovirus construct expressing anti-sense Ku70. (FIG. 10A) Western Blot experiments against Ku70. Cells were infected with a control adenovirus and with the anti-sense Ku70 adenovirus. (FIG. 10B) RNase protection assay of cells infected with the anti-sense Ku70 adenovirus construct. The bars shown correspond to the mRNA levels of each mRNA analyzed from the anti-sense Ku70 infected cells. The mRNA from the control cells is considered 100%.

### DESCRIPTION OF ILLUSTRATIVE EMBODIMENTS

### I. Cardiovascular Disease

Cardiovascular diseases are among the most common natural causes of death. The cardiovascular diseases include many serious diseases which involve the cardiac and vascular systems, such as atherosclerosis, ischemic heart diseases, cardiac failure, cardiac shock, arrhythmia, hypertension, cerebral vascular diseases and peripheral vascular diseases.

Atherosclerosis most often occurs as a complication of hyperlipidemia and can be treated with antihyperlipidemic agents. Ischemic heart disease, cardiac failure, cardiac shock, cerebral vascular disease, peripheral vascular disease, hypertension, arrhythmia and arteriosclerosis may be fatal because ischemia develops in various organs such as the heart, brain and the walls of blood vessels. The ischemia damages the organs in which it develops because it impairs the functions of mitochondria that produce adenosine triphosphate (ATP), which is a phosphate compound with high energy potential serving as an energy source for the constituent cells of these organs. The resulting functional damage of organs can be fatal if it occurs in vital organs such as the heart, brain and blood vessels. It is therefore important for treating these diseases to restore the functional impairment of mitochondria caused by ischemia. Antiarrhythmic agents have been used to treat ischemic heart disease and arrhythmia, but their use with patients with possible cardiac failure has been strictly limited because these agents may cause cardiac arrest by their cardiodepressant effects.

The cardiovascular diseases named above may develop independently, but more often than not they occur in various combinations. For example, ischemic heart diseases are frequently accompanied by arrhythmia and cardiac failure, and complications of cerebrovascular disorder with hypertension are well known. Atherosclerosis is often complicated by one or more cardiovascular diseases and can make the patient seriously ill.

Cardiovascular diseases, which are often complicated by other cardiovascular diseases, have often been treated with a combination of multiple drugs, each of which is specific for a single disease. However, drug-therapy employing multiple agents presents problems for both doctors and patients: doctors always consider compatibilities and contraindications of drugs, and patients suffer both mental and physical distresses due to complicated administration of various drugs and high incidence of adverse reactions. Therefore, it has long been desired to develop a therapeutic agent that has overall pharmacological activities against cardiovascular diseases and which can be employed in the treatment of these diseases with high efficacy.

Ku is a transcription factor that binds to the α-MyHC and down-regulates expression of α-MyHC. As discussed above, upregulation of α-MyHC is indicated for the treatment as well as prevention of cardiovascular diseases. Thus, in accordance with the present invention, methods are described herein for the inhibition of Ku as a method of treating cardiovascular disease.

### A. Hypertrophy, DCM, Chronic Heart Failure

As discussed above, cardiovascular diseases encompass a huge array of syndromes and disorders, all of which combined are among the leading causes of death worldwide. Heart failure by itself is one of the leading causes of morbidity and mortality in the world. In the U.S. alone, estimates indicate that 3 million people are currently living with cardiomyopathy and another 400,000 are diagnosed on a yearly basis. Dilated cardiomyopathy (DCM), also referred to as “congestive cardiomyopathy,” is the most common form of the cardiomyopathies and has an estimated prevalence of nearly 40 per 100,000 individuals (Durand et al., 1995). Although there are other causes of DCM, familiar dilated cardiomyopathy has been indicated as representing approximately 20% of “idiopathic” DCM. Approximately half of the DCM cases are idiopathic, with the remainder being associated with known disease processes. For example, serious myocardial damage can result from certain drugs used in cancer chemotherapy (e.g., doxorubicin and daunoribucin), or from chronic alcohol abuse. Peripartum cardiomyopathy is another idiopathic form of DCM, as is disease associated with infectious sequelae. In sum, cardiomyopathies, including DCM, are significant public health problems.

Heart disease and its manifestations, including coronary artery disease, myocardial infarction, congestive heart failure and cardiac hypertrophy, clearly present a major health risk in the United States today. The cost to diagnose, treat and support patients suffering from these diseases is well into the billions of dollars. Two particularly severe manifestations of heart disease are myocardial infarction and cardiac hypertrophy. With respect to myocardial infarction, typically an acute thrombocytic coronary occlusion occurs in a coronary artery as a result of atherosclerosis and causes myocardial cell death. Because cardiomyocytes, the heart muscle cells, are terminally differentiated and generally incapable of cell division, they are generally replaced by scar tissue when they die during the course of an acute myocardial infarction. Scar tissue is not contractile, fails to contribute to cardiac function, and often plays a detrimental role in heart function by expanding during cardiac contraction, or by increasing the size and effective radius of the ventricle, for example, becoming hypertrophic.

With respect to cardiac hypertrophy, one theory regards this as a disease that resembles aberrant development and, as such, raises the question of whether developmental signals in the heart can contribute to hypertrophic disease. Cardiac hypertrophy is an adaptive response of the heart to virtually all forms of cardiac disease, including those arising from hypertension, mechanical load, myocardial infarction, cardiac arrhythmias, endocrine disorders, and genetic mutations in cardiac contractile protein genes. While the hypertrophic response is initially a compensatory mechanism that augments cardiac output, sustained hypertrophy can lead to DCM, heart failure, and sudden death. In the United States, approximately half a million individuals are diagnosed with heart failure each year, with a mortality rate approaching 50%.

The causes and effects of cardiac hypertrophy have been extensively documented, but the underlying molecular mechanisms have not been elucidated. Understanding these mechanisms is a major concern in the prevention and treatment of cardiac disease and will be crucial as a therapeutic modality in designing new drugs that specifically target cardiac hypertrophy and cardiac heart failure. As pathologic cardiac hypertrophy typically does not produce any symptoms until the cardiac damage is severe enough to produce heart failure, the symptoms of cardiomyopathy are those associated with heart failure. These symptoms include shortness of breath, fatigue with exertion, the inability to lie flat without becoming short of breath (orthopnea), paroxysmal nocturnal dyspnea, enlarged cardiac dimensions, and/or swelling in the lower legs. Patients also often present with increased blood pressure, extra heart sounds, cardiac murmurs, pulmonary and systemic emboli, chest pain, pulmonary congestion, and palpitations. In addition, DCM causes decreased ejection fractions (i.e., a measure of both intrinsic systolic function and remodeling). The disease is further characterized by ventricular dilation and grossly impaired systolic function due to diminished myocardial contractility, which results in dilated heart failure in many patients. Affected hearts also undergo cell/chamber remodeling as a result of the myocyte/myocardial dysfunction, which contributes to the “DCM phenotype.” As the disease progresses so do the symptoms. Patients with DCM also have a greatly increased incidence of life-threatening arrhythmias, including ventricular tachycardia and ventricular fibrillation. In these patients, an episode of syncope (dizziness) is regarded as a harbinger of sudden death.

Diagnosis of dilated cardiomyopathy typically depends upon the demonstration of enlarged heart chambers, particularly enlarged ventricles. Enlargement is commonly observable on chest X-rays, but is more accurately assessed using echocardiograms. DCM is often difficult to distinguish from acute myocarditis, valvular heart disease, coronary artery disease, and hypertensive heart disease. Once the diagnosis of dilated cardiomyopathy is made, every effort is made to identify and treat potentially reversible causes and prevent further heart damage. For example, coronary artery disease and valvular heart disease must be ruled out. Anemia, abnormal tachycardias, nutritional deficiencies, alcoholism, thyroid disease and/or other problems need to be addressed and controlled.

As mentioned above, treatment with pharmacological agents still represents the primary mechanism for reducing or eliminating the manifestations of heart failure. Diuretics constitute the first line of treatment for mild-to-moderate heart failure. Unfortunately, many of the commonly used diuretics (e.g., the thiazides) have numerous adverse effects. For example, certain diuretics may increase serum cholesterol and triglycerides. Moreover, diuretics are generally ineffective for patients suffering from severe heart failure.

If diuretics are ineffective, vasodilatory agents may be used; the angiotensin converting (ACE) inhibitors (e.g., enalapril and lisinopril) not only provide symptomatic relief, they also have been reported to decrease mortality (Young et al., 1989). Again, however, the ACE inhibitors are associated with adverse effects that result in their being contraindicated in patients with certain disease states (e.g., renal artery stenosis). Similarly, inotropic agent therapy (i.e., a drug that improves cardiac output by increasing the force of myocardial muscle contraction) is associated with a panoply of adverse reactions, including gastrointestinal problems and central nervous system dysfunction.

The currently used pharmacological agents have severe shortcomings in particular patient populations. The availability of new, safe and effective agents would undoubtedly benefit patients who either cannot use the pharmacological modalities presently available, or who do not receive adequate relief from those modalities. The prognosis for patients with DCM is variable, and depends upon the degree of ventricular dysfunction, with the majority of deaths occurring within five years of diagnosis.

In light of the limitations of the current therapies, the inventors describe herein the inhibition of the transcription factor Ku as a therapeutic treatment broadly applicable to cardiovascular disease. Inhibiting Ku can lead to the upregulation of α-MyHC, and upregulation of α-MyHC is considered strongly beneficial to the heart and protective as well as responsive to a variety of cardiovascular insults and diseases that lead to decreased cardiac viability

### II. Ku and Alpha Myosin Heavy Chain

As stated before it has been shown that in the non-failing, non-hypertrophied human heart, approximately 20-30% of total MyHC mRNA consists of α-MyHC mRNA whereas in the failing heart, α-MyHC expression represents less than 2% of total MyHC mRNA (Lowes et al., 1997; Nakao et al., 1997). At the protein level, α-MyHC in normal hearts constitutes 7-11% of total MyHC but it is undetectable in the failing heart (Miyata et al., 2000). The importance of α-MyHC expression in the human heart has been emphasized by the finding that mutations in the α-MyHC gene can cause both hypertrophic and dilated cardiomyopathies (Carniel et al., 2003; Niimura et al., 2002). The decrease in α-MyHC in human heart failure may play an important role in the well-established reduction of cardiac contractility.

The promoter proximal region of human α-MyHC is not as well characterized as the rat α-MyHC promoter, but they share ˜80% sequence similarity over a 400 bp region. Based on sequence comparison, the human α-MyHC promoter has putative binding sites for transcription factors that have been shown to be important for the positive regulation of the rat α-MyHC promoter, e.g., GATA4, NFAT3, TEF, thyroid response, MEF2, and SRF, among others (Morkin, 2000). The inventors have shown that the YY1 transcription factor acts as a repressor of the human α-MyHC promoter in cardiac cells and that YY1 levels and DNA binding activity are increased in human heart failure (Sucharov et al., 2003). This was the first report of a transcriptional repressor of the human α-MyHC promoter. YY1 is a 414 amino acid protein that has been shown to activate or repress transcription depending on promoter context or protein interaction (Thomas and Seto, 1999). It has also been shown to function as a polycomb group protein responsible for repression of certain developmentally regulated genes in Drosophila (Atchison et al., 2003).

The down regulation of the α-MyHC gene during heart failure seems likely to contribute to the pathogenesis of the disease (see Abraham et al., 2002). The inventors have shown that YY1 is increased in heart failure and represses the activity of the α-MyHC promoter. The inventors have now identified the Ku factors as additional repressors of the activity of the α-MyHC promoter.

Controversy has surrounded the function of Ku. Ku was first identified as a DNA repair protein that recognizes DNA ends without any preferences for the nature of the ends (Koike, 2002). Once bound to DNA, Ku can interact with the catalytic subunit of the DNA-dependent protein kinase (DNA-PK) and together they constitute the active kinase (Dynan and Yoo, 1998). This complex can phosphorylate several nuclear proteins in vitro, e.g., p53, c-fos, Sp1, XRCC4, DNA-PKcs, or Ku itself and it is involved in the non homologous DNA-end-joining repair and V(D)J recombination (Koike, 2002). Recently Ku has been shown to be important in preventing apoptosis by interacting with Bax and preventing it from entering the mitochondria (Sawada et al., 2003a; 2003b).

At the same time, various reports have shown that Ku can bind DNA in a sequence specific manner as shown in the cases of several genes (Giffin et al., 1999; Camara-Clayette et al., 1999; Kim et al., 1995; Merante et al., 2002; Taranenko and Krause, 2000; Willis et al., 2002). Recent reports showed that the Ku70/80 complex interacts with different transcription factors, e.g., heat shock 19 factor, DNA binding domain of the progesterone receptors and homeodomain proteins (Sartorious et al., 2000; Willis et al., 2002; Schild-Poulter et al., 2001; Schaffer et al., 2003; Ko and Chin, 2003), suggesting that this could be a mechanism by which Ku is recruited to specific regions of promoters/enhancers. Ku has also been shown to interact with RNA polymerase II and with TBP (Tuteja and Tuteja, 2000). As shown herein, the inventors' experiments suggest that binding of Ku to the α-MyHC promoter is sequence-specific.

As discussed above, Ku has been recently shown to interact with different transcription factors. At the same time, YY1 has been shown to be involved in DNA repair though the interaction with PARP-1 (Oei and Shi, 2001a; 2001b). Ku is also known to interact with PARP-1, and immunoprecipitation experiments using YY1, Ku70 or Ku80 antibodies suggest that these proteins interact in cells (see Examples supra). The consequences of this interaction are important to the understanding of Ku's function as a transcription factor and of YY1's function as a protein involved in repair and vice versa. It has been recently proposed that Ku is involved in the re-initiation phase of transcription and that there would be an equilibrium between re-initiation and repair (Woodard et al., 2001). In this model, Ku would be either sequestered in a Ku-dependent re-initiation complex, where it would not be capable of interacting with DNA ends or, in the presence of DNA damage signals, Ku and DNA-PKcs would be released from this complex and become active for repair. This would, in turn, disrupt the transcription apparatus, preventing re-initiation from occurring. YY1 has, in turn, been shown to be important for transcription initiation and Usheva and Shenk (1994) have shown in vitro that YY1, TFIIB and RNA polymerase II are sufficient to initiate transcription of the AAV P5 promoter. The inventors now propose that YY1 and Ku are part of a transcription complex that can be involved in DNA repair or transcription and that their function will vary according to the integrity of the DNA.

Ku has been shown to function either as a repressor (Bliss and Lane, 1997; Dignam et al., 1983; Giffin et al., 1996) or as an activator (Shintani et al., 2003) in transient transfection experiments. It is extremely interesting that both YY1 and Ku levels are increased in heart failure. Finally, Ku has been shown to be expressed in various organisms besides human, including monkey, Xenopus, yeast, Drosophila and rodents (Bliss and Lane, 1997). Interestingly, Ku levels in rodents are 21-fold decreased in comparison to humans (Bliss and Lane, 1997). At the same time, α-MyHC mRNA levels are increased in rodents when compared to humans. One can speculate that the difference in the levels of α-MyHC and Ku are related and part of an evolutionary process. Ku thus presents as a target molecule present in a wide variety of species that is also intimately linked to the cardiovascular disease process. Inhibiting the action of Ku could potentially reverse or alleviate the damage done by the downregulation of α-MyHC as well as indirectly (or even directly) altering the cellular cascades that lead to heart failure and hypertrophy.

### III. Cellular Components of Heart Disease

### A. Calcineurin

Calcineurin is a ubiquitously expressed serine/threonine phosphatase that exists as a heterodimer, comprised of a 59 kD calmodulin-binding catalytic A subunit and a 19 kD Ca(++)-binding regulatory B subunit (Stemmer and Klee, 1994; Su et al., 1995). Calcineurin is uniquely suited to mediate the prolonged hypertrophic response of a cardiomyocyte to Ca(++) signaling because the enzyme is activated by a sustained Ca(++) plateau and is insensitive to transient Ca(++) fluxes as occur in response to cardiomyocytc contraction (Dolmetsch et al., 1997).

Activation of calcineurin is mediated by binding of Ca(++) and calmodulin to the regulatory and catalytic subunits, respectively. Previous studies showed that over-expression of calmodulin in the heart also results in hypertrophy, but the mechanism involved was not determined (Gruver et al., 1993). It is now clear that calmodulin acts through the calcineurin pathway to induce the hypertrophic response. Calcineurin has been shown previously to phosphorylate NF-AT3, which subsequently acts on the transcription factor MEF-2 (Olson et al., 1995). Once this event occurs, MEF-2 activates a variety of genes known as fetal genes, the activation of which inevitably results in hypertrophy (see below).

CsA and FK-506 bind the immunophilins cyclophilin and FK-506-binding protein (FKBP12), respectively, forming complexes that bind the calcineurin catalytic subunit and inhibit its activity. CsA and FK-506 block the ability of cultured cardiomyocytes to undergo hypertrophy in response to AngII and PE. Both of these hypertrophic agonists have been shown to act by elevating intracellular Ca(++), which results in activation of the PKC and MAP kinase signaling pathways (Sadoshima et al., 1993; Sadoshima and Izumo, 1993; Kudoh et al., 1997; Yamazaki et al., 1997, Zou et al., 1996). CsA does not interfere with early signaling events at the cell membrane, such as PI turnover, Ca(++) mobilization, or PKC activation (Emmel et al., 1989). Thus, its ability to abrogate the hypertrophic responses of AngII and PE suggests that calcineurin activation is an essential step in the AngII and PE signal transduction pathways, and its action has been shown to be mediated through transcription factor NF-AT3.

### B. NF-AT3

NF-AT3 is a member of a multigene family containing four members, NF-ATc, NF-ATp, NF-AT3, and NF-AT4 (McCaffery et al., 1993; Northrup et al., 1994; Hoey et al., 1995; Masuda et al., 1995; Park et al., 1996; Ho et al., 1995). These factors bind the consensus DNA sequence GGAAAAT as monomers or dimers through a Rel homology domain (RHD) (Rooney et al., 1994; Hoey et al., 1995). Three of the NF-AT genes are restricted in their expression to T-cells and skeletal muscle, whereas NF-AT3 is expressed in a variety of tissues including the heart (Hoey et al., 1995). For additional disclosure regarding NF-AT proteins the skilled artisan is referred to U.S. Pat. No. 5,708,158, specifically incorporated herein by reference.

NF-AT3 is a 902-amino acid protein with a regulatory domain at its amino-terminus that mediates nuclear translocation and the Rel-homology domain near its carboxyl-terminus that mediates DNA binding. There are three different steps involved in the activation of NF-AT proteins, namely, dephosphorylation, nuclear localization and an increase in affinity for DNA. In resting cells, NFAT proteins are phosphorylated and reside in the cytoplasm. These cytoplasmic NF-AT proteins show little or no DNA affinity. Stimuli that elicit calcium mobilization result in the rapid dephosphorylation of the NF-AT proteins and their translocation to the nucleus. The dephosphorylated NF-AT proteins show an increased affinity for DNA. Each step of the activation pathway may be blocked by CsA or FK506. This implies, and earlier studies have shown, that calcineurin is the protein responsible for NF-AT activation (Olson et al., 1995).

Thus, many of the changes in gene expression in response to calcineurin activation are mediated by members of the NF-AT family of transcription factors, which translocate to the nucleus following dephosphorylation by calcineurin. Many observations support the conclusion that NF-AT also is an important mediator of cardiac hypertrophy in response to calcineurin activation. NF-AT activity is induced by treatment of cardiomyocytes with AngII and PE. This induction is blocked by CsA and FK-506, indicating that it is calcineurin-dependent. NF-AT3 synergizes with GATA4 to activate the cardiac specific BNP promoter in cardiomyocytes. Also, expression of activated NF-AT3 in the heart is sufficient to bypass all upstream elements in the hypertrophic signaling pathway and evoke a hypertrophic response.

Prior work demonstrates that the C-terminal portion of the Rel-homology domain of NF-AT3 interacts with the second zinc finger of GATA4, as well as with GATA5 and GATA6, which are also expressed in the heart. The crystal structure of the DNA binding region of NF-ATc has revealed that the C-terminal portion of the Rel-homology domain projects away from the DNA binding site and also mediates interaction with AP-1 in immune cells (Wolfe et al., 1997).

According to one model previously proposed, hypertrophic stimuli such as AngII and PE, which lead to an elevation of intracellular Ca(++), result in activation of calcineurin. NF-AT3 within the cytoplasm is dephosphorylated by calcineurin, enabling it to translocate to the nucleus where it can interact with GATA4, and then activate the transcription factor MEF-2, a family of transcription factors that are normally repressed by a tight association with class II HDAC's.

Results of previous work have shown that calcineurin activation of NF-AT3 regulates hypertrophy in response to a variety of pathologic stimuli and suggests a sensing mechanism for altered sarcomeric function. Of note, there are several familial hypertrophic cardiomyopathies (FHC) caused by mutations in contractile protein genes, which result in subtle disorganization in the fine crystalline-like structure of the sarcomere (Watkins et al., 1995; Vikstrom and Leinwand, 1996). It is unknown how sarcomeric disorganization is sensed by the cardiomyocyte, but it is apparent that this leads to altered Ca(++) handling (Palmiter and Solaro, 1997; Botinelli et al., 1997; Lin et al., 1996). Calcineurin, as discussed above, is one of the sensing molecules that couples altered Ca(++) handling associated with FHC with cardiac hypertrophy and heart failure. As has been mentioned previously, these studies and the relation between NF-AT3 and calcineurin led to a search for molecules or agents that could modulate calcineurin's activation of NF-AT3 specifically in cardiac cells.

### C. MCIP-1

The importance of MCIP was unraveled during the aforementioned efforts to discover modulators of calcineurin in relation to calcineurin's role in heart failure and cardiovascular diseases such as hypertrophy. One class of endogenous calcineurin inhibitors are the modulatory calcineurin-interacting proteins MCIP1, 2 and 3 (previously known as DSCR1, ZAKI-4 and DSCR1L), a family of inhibitory proteins expressed primarily in striated muscle and nervous tissue (reviewed in Rothermel et al., 2003). MCIP-1 is unique among endogenous calcineurin inhibitors in that activated calcineurin strongly induces expression of a splice variant of MCIP-1 mRNA in transgenic mouse hearts and cultured myocytes, suggesting that MCIP-1 protein functions as a feedback inhibitor, protecting the cardiac myocyte from unchecked calcineurin activity.

Enhanced MCIP-1 expression may be a common response of the heart to a variety of hypertrophic stimuli, since pressure overload, mechanical strain, and hypertrophic agonists have all been demonstrated to increase cardiac expression of MCIP-1 mRNA. Furthermore, overexpression of MCIP-1 in the hearts of transgenic mice attenuated the hypertrophic response induced by activated calcineurin, β-adrenergic stimulation, exercise training, pressure overload and myocardial infarction, supporting a role for calcineurin-dependent signaling in diverse forms of cardiac hypertrophy.

MCIP-1 directly binds and inhibits calcineurin, functioning as an endogenous feedback inhibitor of calcineurin activity. Overexpression of MCIP-1 in the hearts of transgenic animals is anti-hypertrophic; MCIP-1 attenuates in vivo models of both calcineurin-dependent hypertrophy (Rothermel et al., 2001) and pressure-overload-induced hypertrophy (Hill et al., 2002). MCIP-1 also acts as a substrate for phosphorylation by MAPK and GSK-3, and calcineurin's phosphatase activity. Residues 81-177 of MCIP-1 retain the calcineurin inhibitory action.

Binding of MCIP-1 to calcineurin does not require calmodulin, nor does MCIP-1 interfere with calmodulin binding to calcineurin. This suggests that the surface of calcineurin to which MCIP-1 bindings does not include the calmodulin binding domain. In contrast, the interaction of MCIP-1 with calcineurin is disrupted by FK506:FKBP or cyclosporin:cyclophylin, indicating that the surface of calcineurin to which MCIP-1 binds overlaps with that required for the activity of immunosuppressive drugs.

### D. MEF2

As mentioned above, NF-AT3 activation by Calcineurin leads to the activation of another family of transcription factors, the myocyte enhancer factor-2 family (MEF2), which are known to play an important role in morphogenesis and myogenesis of skeletal, cardiac, and smooth muscle cells (Olson et al., 1995). Thus, inhibition of calcineurin through MCIP-1 would likely alter or abrogate the activation of MEF2, explaining at least in part the anti-hypertrophic properties of MCIP-1.

MEF2 factors are expressed in all developing muscle cell types, binding a conserved DNA sequence in the control regions of the majority of muscle-specific genes. Of the four mammalian MEF2 genes, three (MEF2A, MEF2B and MEF2C) can be alternatively spliced, which have significant functional differences (Brand, 1997; Olson et al., 1995). These transcription factors share homology in an N-terminal MADS-box and an adjacent motif known as the MEF2 domain. Together, these regions of MEF2 mediate DNA binding, homo- and heterodimerization, and interaction with various cofactors, such as the myogenic bHLH proteins in skeletal muscle. Additionally, biochemical and genetic studies in vertebrate and invertebrate organisms have demonstrated that MEF2 factors regulate myogenesis through combinatorial interactions with other transcription factors.

Loss-of-function studies indicate that MEF2 factors are essential for activation of muscle gene expression during embryogenesis. The expression and functions of MEF2 proteins are subject to multiple forms of positive and negative regulation, serving to fine-tune the diverse transcriptional circuits in which the MEF2 factors participate. MEF-2 is bound in an inactive form in the healthy heart by class II HDACS (see supra), and when MEF-2 is activated it is released from the HDAC and activates the fetal gene program that is so deleterious for the heart.

### E. Histone Deacetylase

Nucleosomes, the primary scaffold of chromatin folding, are dynamic macromolecular structures, influencing chromatin solution conformations (Workman and Kingston, 1998). The nucleosome core is made up of histone proteins, H2A, HB, H3 and H4. Histone acetylation causes nucleosomes and nucleosomal arrangements to behave with altered biophysical properties. The balance between activities of histone acetyl transferases (HAT) and deacetylases (HDAC) determines the level of histone acetylation. Acetylated histones cause relaxation of chromatin and activation of gene transcription, whereas deacetylated chromatin generally is transcriptionally inactive.

More than seventeen different HDACs have been cloned from vertebrate organisms. The first three human HDACs identified were HDAC 1, HDAC 2 and HDAC 3 (termed class I human HDACs), and HDAC 8 (Van den Wyngaert et al., 2000). Class II human HDACs, HDAC 4, HDAC 5, HDAC 6, HDAC 7, HDAC 9, and HDAC 10 (Kao et al., 2000) have been cloned and identified (Grozinger et al., 1999; Zhou et al. 2001; Tong et al., 2002). Additionally, HDAC 11 has been identified but not yet classified as either class I or class II (Gao et al., 2002) and there is a new class of HDACs known as class III. HDACs 4, 5, 7, 9 and 10 have a unique amino-terminal extension not found in other HDACs. This amino-terminal region contains the MEF2-binding domain. HDACs 4, 5 and 7 have been shown to be involved in the regulation of cardiac gene expression and in particular embodiments, repressing MEF2 transcriptional activity. The exact mechanism in which class II HDACs repress MEF2 activity is not completely understood. One possibility is that HDAC binding to MEF2 inhibits MEF2 transcriptional activity, either competitively or by destabilizing the native, transcriptionally active MEF2 conformation. It also is possible that class II HDACs require dimerization with MEF2 to localize or position HDAC in a proximity to histones for deacetylation to proceed. No matter how HDACs inhibit MEF-2, calcium signaling mediated through calcineurin is responsible for freeing HDACs from MEF-2, leading to activation of the fetal gene program.

A variety of inhibitors for histone deacetylase have been identified. The proposed uses range widely, but primarily focus on cancer therapy. See Saunders et al. (1999); Jung et al. (1997); Jung et al. (1999); Vigushin et al. (1999); Kim et al. (1999); Kitazomo et al. (2001); Vigushin et al. (2001); Hoffmann et al. (2001); Kramer et al. (2001); Massa et al (2001); Komatsu et al. (2001); Han et al. (2000). Such therapy is the subject of NIH sponsored clinical trials for solid and hematological tumors. HDAC's also increase transcription of transgenes, thus constituting a possible adjunct to gene therapy. Yamano et al. (2000); Su et al. (2000).

HDACs can be inhibited through a variety of different mechanisms—proteins, peptides, and nucleic acids (including antisense, RNAi molecules, and ribozymes). Methods are widely known to those of skill in the art for the cloning, transfer and expression of genetic constructs, which include viral and non-viral vectors, and liposomes. Viral vectors include adenovirus, adeno-associated virus, retrovirus, vaccina virus and herpesvirus.

Perhaps the most widely known small molecule inhibitor of HDAC function is Trichostatin A, a hydroxamic acid. It has been shown to induce hyperacetylation and cause reversion of ras transformed cells to normal morphology (Taunton et al., 1996) and induces immunsuppression in a mouse model (Takahashi et al., 1996). It is commercially available from a variety of sources including BIOMOL Research Labs, Inc., Plymouth Meeting, Pa.

The following references, incorporated herein by reference, all describe HDAC inhibitors that may find use in the present invention: AU 9,013,101; AU 9,013,201; AU 9,013,401; AU 6,794,700; EP 1,233,958; EP 1,208,086; EP 1,174,438; EP 1,173,562; EP 1,170,008; EP 1,123,111; JP 2001/348340; U.S. 2002/256221; U.S. 2002/103192; U.S. 2002/65282; U.S. 2002/61860; WO 02/51842; WO 02/50285; WO 02/46144; WO 02/46129; WO 02/30879; WO 02/26703; WO 02/26696; WO 01/70675; WO 01/42437; WO 01/38322; WO 01/18045; WO 01/14581; Furumai et al. (2002); Hinnebusch et al. (2002); Mai et al. (2002); Vigushin et al. (2002); Gottlicher et al. (2001); Jung (2001); Komatsu et al. (2001); Su et al. (2000).

### IV. Methods of Treating Heart Disease

Heart disease of some forms may be curable and these are dealt with by treating the primary disease, such as anemia or thyrotoxicosis. Also curable are forms caused by anatomical problems, such as a heart valve defect. These defects can be surgically corrected. However, for the most common forms of heart failure no known cure exists. Treating the symptoms of these diseases helps, and some treatments of the disease have been successful. The treatments attempt to improve patients' quality of life and length of survival through lifestyle change and drug therapy.

Patients can minimize the effects of heart failure by controlling the risk factors for heart disease, but even with lifestyle changes, most heart failure patients must take medication, many of whom receive two or more drugs.

Several types of drugs have proven useful in the treatment of heart failure: Diuretics help reduce the amount of fluid in the body and are useful for patients with fluid retention and hypertension; and digitalis can be used to increase the force of the heart's contractions, helping to improve circulation. Results of several earlier studies placed a lot of emphasis on the use of ACE inhibitors (Manoria and Manoria, 2003). Several large studies have indicated that ACE inhibitors improve survival among heart failure patients and may slow, or perhaps even prevent, the loss of heart pumping activity (for a review see De Feo et al., 2003; DiBianco, 2003). Patients who cannot take ACE inhibitors may get a nitrate and/or a drug called hydralazine, each of which helps relax tension in blood vessels to improve blood flow (Ahmed, 2003).

Heart failure is almost always life-threatening. When drug therapy and lifestyle changes fail to control its symptoms, a heart transplant may be the only treatment option. However, candidates for transplantation often have to wait months or even years before a suitable donor heart is found. Recent studies indicate that some transplant candidates improve during this waiting period through drug treatment and other therapy, and can be removed from the transplant list (Conte et al., 1998).

Transplant candidates who do not improve sometimes need mechanical pumps, which are attached to the heart. Called left ventricular assist devices (LVADs), the machines take over part or virtually all of the heart's blood-pumping activity. However, current LVADs are not permanent solutions for heart failure but are considered bridges to transplantation.

As a final alternative, there is an experimental surgical procedure for severe heart failure available called cardiomyoplasty. (Dumcius et al., 2003) This procedure involves detaching one end of a muscle in the back, wrapping it around the heart, and then suturing the muscle to the heart. An implanted electric stimulator causes the back muscle to contract, pumping blood from the heart. To date, none of these treatments have been shown to cure heart failure, but can at least improve quality of life and extend life for those suffering this disease.

As with heart failure, there are no known cures to hypertrophy. Current medical management of cardiac hypertrophy, in the setting of a cardiovascular disorder includes the use of at least two types of drugs: inhibitors of the rennin-angiotensoin system, and β-adrenergic blocking agents (Bristow, 1999). Therapeutic agents to treat pathologic hypertrophy in the setting of heart failure include angiotensin II converting enzyme (ACE) inhibitors and β-adrenergic receptor blocking agents (Eichhorn and Bristow, 1996). Other pharmaceutical agents that have been disclosed for treatment of cardiac hypertrophy include angiotensin II receptor antagonists (U.S. Pat. No. 5,604,251) and neuropeptide Y antagonists (PCT Publication No. WO 98/33791).

Non-pharmacological treatment is primarily used as an adjunct to pharmacological treatment. One means of non-pharmacological treatment involves reducing the sodium in the diet. In addition, non-pharmacological treatment also entails the elimination of certain precipitating drugs, including negative inotropic agents (e.g., certain calcium channel blockers and antiarrhythmic drugs like disopyramide), cardiotoxins (e.g., amphetamines), and plasma volume expanders (e.g., nonsteroidal anti-inflammatory agents and glucocorticoids).

As can be seen from the discussion above, there is a great need for a successful treatment approach to heart failure and hypertrophy. Thus, in one embodiment of the present invention, methods for the treatment of cardiac hypertrophy or heart failure utilizing inhibitors of Ku are provided. For the purposes of the present application, treatment comprises reducing one or more of the symptoms of heart failure or cardiac hypertrophy, such as reduced exercise capacity, reduced blood ejection volume, increased left ventricular end diastolic pressure, increased pulmonary capillary wedge pressure, reduced cardiac output, cardiac index, increased pulmonary artery pressures, increased left ventricular end systolic and diastolic dimensions, and increased left ventricular wall stress, wall tension and wall thickness-same for right ventricle. In addition, use of inhibitors of Ku may prevent cardiac hypertrophy and its associated symptoms from arising.

### A. Pharmaceutical Inhibitors

Inhibiting Ku as a pharmaceutical treatment has only recently begun to be studied and as such, no commercial compounds have been discovered or described. Identification of a pharmaceutical or small molecule inhibitor of Ku can be readily accomplished through standard high-throughput screening methods. One can screen large compound libraries using the screens described herein to discover small molecules capable of inhibiting Ku. Furthermore, standard medicial chemistry approaches can be applied to these compounds to enhance or modify their activity so as to yield additional compounds.

### B. Antisense Constructs

An alternative approach to inhibiting Ku would be utilization of antisense technology. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit or promote gene transcription or translation or both within a host cell, either in vitro or in vivo, such as within a host animal, including a human subject. Repression of gene transcription may lead to downregulation or inhibition of Ku, while promotion of gene transcription could activate the transcription of a repressor gene that controls Ku expression. Thus, one of skill can easily envision ways in which antisense could be used to hinder Ku expression.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

### C. Ribozymes

Another general class of inhibitors of Ku would be Ku RNA-specific ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cook, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cook et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cook et al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). It has also been shown that ribozymes can elicit genetic changes in some cells lines to which they were applied; the altered genes included the oncogenes H-ras, c-fos and genes of HIV. Most of this work involved the modification of a target mRNA, based on a specific mutant codon that was cleaved by a specific ribozyme.

### D. RNAi (siRNA)

RNA interference (also referred to as “RNA-mediated interference” or RNAi) is another mechanism by which Ku expression could be modulated in a way similar to that of the antisense methodology. One can envision instances when Ku RNAs could be reduced or eliminated, leading to decreased expression of Ku. Double-stranded RNA (dsRNA) has been observed to mediate the reduction, which is a multi-step process. dsRNA activates post-transcriptional gene expression surveillance mechanisms that appear to function to defend cells from virus infection and transposon activity (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi (or siRNA) offers major experimental advantages for study of gene function. These advantages include a very high specificity, ease of movement across cell membranes, and prolonged down-regulation of the targeted gene (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above (Montgomery et al., 1998).

The making of siRNAs has been mainly through direct chemical synthesis; through processing of longer, double-stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single-stranded RNA-oligomers followed by the annealing of the two single-stranded oligomers into a double-stranded RNA. Methods of chemical synthesis are diverse. Non-limiting examples are provided in U.S. Pat. Nos. 5,889,136, 4,415,732, and 4,458,066, expressly incorporated herein by reference, and in Wincott et al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides+3′ non-complementary dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et al. (2001) wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et al., 2000; Elbashir et al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g., T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single-strands of RNA can be produced enzymatically or by partial/total organic synthesis. Preferably, single-stranded RNA is enzymatically synthesized from the PCR products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

### E. Antibodies

In certain aspects of the invention, antibodies may find use as antagonists of Ku activity or expression. As used herein, the term “antibody” is intended to refer broadly to any appropriate immunologic binding agent such as IgG, IgM, IgA, IgD and IgE. Generally, IgG and/or IgM are preferred because they are the most common antibodies in the physiological situation and because they are most easily made in a laboratory setting.

The term “antibody” also refers to any antibody-like molecule that has an antigen binding region, and includes antibody fragments such as Fab′, Fab, F(ab′)₂, single domain antibodies (DABs), Fv, scFv (single chain Fv), and the like. The techniques for preparing and using various antibody-based constructs and fragments are well known in the art.

Monoclonal antibodies (MAbs) are recognized to have certain advantages, e.g., reproducibility and large-scale production, and their use is generally preferred. The invention thus provides monoclonal antibodies of the human, murine, monkey, rat, hamster, rabbit and even chicken origin. Due to the ease of preparation and ready availability of reagents, murine monoclonal antibodies will often be preferred.

Single-chain antibodies are described in U.S. Pat. Nos. 4,946,778 and 5,888,773, each of which are hereby incorporated by reference.

“Humanized” antibodies are also contemplated, as are chimeric antibodies from mouse, rat, or other species, bearing human constant and/or variable region domains, bispecific antibodies, recombinant and engineered antibodies and fragments thereof. Methods for the development of antibodies that are “custom-tailored” to the patient's disease are likewise known and such custom-tailored antibodies are also contemplated.

### F. Peptide Aptamers, Peptoids and Other Mimetics

Peptide aptamers represent yet another potential mechanism for either inhibiting Ku or disturbing/treating a secondary signaling cascade related to or involved in cardiac hypertrophy or heart failure. Recently, the ability to manipulate individual genes has driven the development of reverse genetics, in which the function of genes is inferred from the phenotypes that arise from their mutation. In diploids, reverse genetics also typically requires generation of homozygotes in the mutated gene. To circumvent this requirement, a number of dominant “reverse genetic” methods to inactivate gene function have been devised, including inhibition by drugs, expression of dominant-negative proteins, injection of antibodies, expression of antisense RNAs, expression of nucleic acid aptamers, and expression of peptide aptamers (Geyer et al., 1999).

The ability to specifically interfere with the function of proteins of pathological significance has been a goal for molecular medicine for many years. Peptide aptamers are proteins that contain a conformationally constrained peptide region of variable sequence displayed from a scaffold (Geyer et al., 1999). Peptide aptamers comprise a new class of molecules, with a peptide moiety of randomized sequence, which are selected for their ability to bind to a given target protein under intracellular conditions (Hoppe-Seyler et al., 2004). They have the potential to inhibit the biochemical activities of a target protein, can delineate the interactions of the target protein in regulatory networks, and identify novel therapeutic targets. Peptide aptamers represent a new basis for drug design and protein therapy, with implications for basic and applied research, for a broad variety of different types of diseases (Hoppe-Seyler et al., 2004).

Peptide aptamers from combinatorial libraries can be dominant inhibitors of gene function. Researchers have used two-hybrid systems to select aptamers based on Escherichia coli thioredoxin (TrxA) that recognize specific proteins and allelic variants. Apterms have been selected against Cdk2 (Colas et al., 1996), Ras (Xu et al, 1997), E2F (Fabbrizio et al., 1999), and HIV-1 Rev (Cohen et al., 1998). Apterms have been used in mammalian cells (Cohen et al, 1998) and in Drosophila melanogaster (Kolonin et al., 1998). These recent results demonstrate the power and potential utility of peptide aptamers, both as stand-alone therapeutics and even as a potential class of inhibitors of nuclear export. As such they could be used to block nuclear export, or they could be used in conjunction with an inhibitor of nuclear export as a dual or combination therapy.

Polypeptoids, or poly-N-substituted glycines, similar to polypeptides in structure, are comprised of N-substituted glycine monomers. Peptoids are amino acids in which the side chain is bonded to the nitrogen atom instead of the alpha-carbon. Peptoid synthesis may provide a less expensive surfactant replacement. The constitute yet another type of “mimetic” or mimic stucture that present similar features as a given polypeptide.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins. Vita et al. (1998). A structural motif that appears to be evolutionarily conserved in certain toxins is small (30-40 amino acids), stable, and high permissive for mutation. This motif is composed of a beta sheet and an alpha helix bridged in the interior core by three disulfides.

Beta II turns have been mimicked successfully using cyclic L-pentapeptides and those with D-amino acids. Weisshoff et al. (1999). Also, Johannesson et al. (1999) report on bicyclic tripeptides with reverse turn inducing properties.

Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

### H. Combined Therapy

In another embodiment, it is envisioned to use an inhibitor of Ku in combination with other therapeutic modalities. Thus, in addition to the therapies described above, one may also provide to the patient more “standard” pharmaceutical cardiac therapies. Examples of other therapies include, without limitation, so-called “beta blockers,” anti-hypertensives, cardiotonics, anti-thrombotics, vasodilators, hormone antagonists, iontropes, diuretics, endothelin antagonists, calcium channel blockers, phosphodiesterase inhibitors, ACE inhibitors, angiotensin type 2 antagonists and cytokine blockers/inhibitors, and HDAC inhibitors.

Combinations may be achieved by contacting cardiac cells with a single composition or pharmacological formulation that includes both agents, or by contacting the cell with two distinct compositions or formulations, at the same time, wherein one composition includes the expression construct and the other includes the agent. Alternatively, the therapy using an inhibitor of Ku may precede or follow administration of the other agent(s) by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either an inhibitor of Ku or the other agent will be desired. In this regard, various combinations may be employed. By way of illustration, where the Ku inhibitor is “A” and the other agent is “B,” the following permutations based on 3 and 4 total administrations are exemplary:

A/B/A B/A/B B/B/A A/A/B B/A/A A/B/B B/B/B/A
B/B/A/B A/A/B/B A/B/A/B A/B/B/A B/B/A/A B/A/B/A
B/A/A/B B/B/B/A A/A/A/B B/A/A/A A/B/A/A A/A/B/A
A/B/B/B B/A/B/B B/B/A/B

Other combinations are likewise contemplated.

I. Adjunct Therapeutic Agents

Pharmacological therapeutic agents and methods of administration, dosages, etc., are well known to those of skill in the art (see for example, the “Physicians Desk Reference,” Goodman & Gilman's “The Pharmacological Basis of Therapeutics,” “Remington's Pharmaceutical Sciences,” and “The Merck Index, Thirteenth Edition,” incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such invidual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, an antianginal agent, an antibacterial agent or a combination thereof.

In addition, it should be noted that any of the following may be used to develop new sets of cardiac therapy target genes as β-blockers were used in the present examples (see below). While it is expected that many of these genes may overlap, new gene targets likely can be developed.

1. Antihyperlipoproteinemics

In certain embodiments, administration of an agent that lowers the concentration of one of more blood lipids and/or lipoproteins, known herein as an “antihyperlipoproteinemic,” may be combined with a cardiovascular therapy according to the present invention, particularly in treatment of athersclerosis and thickenings or blockages of vascular tissues. In certain aspects, an antihyperlipoproteinemic agent may comprise an aryloxyalkanoic/fibric acid derivative, a resin/bile acid sequesterant, an HMG CoA reductase inhibitor, a nicotinic acid derivative, a thyroid hormone or thyroid hormone analog, a miscellaneous agent or a combination thereof.

a. Aryloxyalkanoic Acid/Fibric Acid Derivatives

Non-limiting examples of aryloxyalkanoic/fibric acid derivatives include beclobrate, enzafibrate, binifibrate, ciprofibrate, clinofibrate, clofibrate (atromide-S), clofibric acid, etofibrate, fenofibrate, gemfibrozil (lobid), nicofibrate, pirifibrate, ronifibrate, simfibrate and theofibrate.

b. Resins/Bile Acid Sequesterants

Non-limiting examples of resins/bile acid sequesterants include cholestyramine (cholybar, questran), colestipol (colestid) and polidexide.

c. HMG CoA Reductase Inhibitors

Non-limiting examples of HMG CoA reductase inhibitors include lovastatin (mevacor), pravastatin (pravochol) or simvastatin (zocor).

d. Nicotinic Acid Derivatives

Non-limiting examples of nicotinic acid derivatives include nicotinate, acepimox, niceritrol, nicoclonate, nicomol and oxiniacic acid.

e. Thryroid Hormones and Analogs

Non-limiting examples of thyroid hormones and analogs thereof include etoroxate, thyropropic acid and thyroxine.

f. Miscellaneous Antihyperlipoproteinemics

Non-limiting examples of miscellaneous antihyperlipoproteinemics include acifran, azacosterol, benfluorex, b-benzalbutyramide, carnitine, chondroitin sulfate, clomestrone, detaxtran, dextran sulfate sodium, 5,8,11,14,17-eicosapentaenoic acid, eritadenine, furazabol, meglutol, melinamide, mytatrienediol, ornithine, g-oryzanol, pantethine, pentaerythritol tetraacetate, a-phenylbutyramide, pirozadil, probucol (lorelco), b-sitosterol, sultosilic acid-piperazine salt, tiadenol, triparanol and xenbucin.

2. Antiarteriosclerotics

Non-limiting examples of an antiarteriosclerotic include pyridinol carbamate.

3. Antithrombotic/Fibrinolytic Agents

In certain embodiments, administration of an agent that aids in the removal or prevention of blood clots may be combined with administration of a modulator, particularly in treatment of athersclerosis and vasculature (e.g., arterial) blockages. Non-limiting examples of antithrombotic and/or fibrinolytic agents include anticoagulants, anticoagulant antagonists, antiplatelet agents, thrombolytic agents, thrombolytic agent antagonists or combinations thereof.

In certain aspects, antithrombotic agents that can be administered orally, such as, for example, aspirin and wafarin (coumadin), are preferred.

a. Anticoagulants

A non-limiting example of an anticoagulant include acenocoumarol, ancrod, anisindione, bromindione, clorindione, coumetarol, cyclocumarol, dextran sulfate sodium, dicumarol, diphenadione, ethyl biscoumacetate, ethylidene dicoumarol, fluindione, heparin, hirudin, lyapolate sodium, oxazidione, pentosan polysulfate, phenindione, phenprocoumon, phosvitin, picotamide, tioclomarol and warfarin.

b. Antiplatelet Agents

Non-limiting examples of antiplatelet agents include aspirin, a dextran, dipyridamole (persantin), heparin, sulfinpyranone (anturane) and ticlopidine (ticlid).

c. Thrombolytic Agents

Non-limiting examples of thrombolytic agents include tissue plasminogen activator (activase), plasmin, pro-urokinase, urokinase (abbokinase) streptokinase (streptase), anistreplase/APSAC (eminase).

4. Blood Coagulants

In certain embodiments wherein a patient is suffering from a hemhorrage or an increased likelyhood of hemhorraging, an agent that may enhance blood coagulation may be used. Non-limiting examples of a blood coagulation promoting agent include thrombolytic agent antagonists and anticoagulant antagonists.

a. Anticoagulant Antagonists

Non-limiting examples of anticoagulant antagonists include protamine and vitamine K1.

b. Thrombolytic Agent Antagonists and Antithrombotics

Non-limiting examples of thrombolytic agent antagonists include amiocaproic acid (amicar) and tranexamic acid (amstat). Non-limiting examples of antithrombotics include anagrelide, argatroban, cilstazol, daltroban, defibrotide, enoxaparin, fraxiparine, indobufen, lamoparan, ozagrel, picotamide, plafibride, tedelparin, ticlopidine and triflusal.

5. Antiarrhythmic Agents

Non-limiting examples of antiarrhythmic agents include Class I antiarrhythmic agents (sodium channel blockers), Class II antiarrhythmic agents (beta-adrenergic blockers), Class II antiarrhythmic agents (repolarization prolonging drugs), Class IV antiarrhythmic agents (calcium channel blockers) and miscellaneous antiarrhythmic agents.

a. Sodium Channel Blockers

Non-limiting examples of sodium channel blockers include Class IA, Class IB and Class IC antiarrhythmic agents. Non-limiting examples of Class IA antiarrhythmic agents include disppyramide (norpace), procainamide (pronestyl) and quinidine (quinidex). Non-limiting examples of Class IB antiarrhythmic agents include lidocaine (xylocalne), tocainide (tonocard) and mexiletine (mexitil). Non-limiting examples of Class IC antiarrhythmic agents include encainide (enkaid) and flecainide (tambocor).

b. Beta Blockers

Non-limiting examples of a beta blocker, otherwise known as a β-adrenergic blocker, a β-adrenergic antagonist or a Class II antiarrhythmic agent, include acebutolol (sectral), alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilolol, carazolol, carteolol, carvedilol, celiprolol, cetamolol, cloranolol, dilevalol, epanolol, esmolol (brevibloc), indenolol, labetalol, levobunolol, mepindolol, metipranolol, metoprolol, moprolol, nadolol, nadoxolol, nifenalol, nipradilol, oxprenolol, penbutolol, pindolol, practolol, pronethalol, propanolol (inderal), sotalol (betapace), sulfinalol, talinolol, tertatolol, timolol, toliprolol and xibinolol. In certain aspects, the beta blocker comprises an aryloxypropanolamine derivative. Non-limiting examples of aryloxypropanolamine derivatives include acebutolol, alprenolol, arotinolol, atenolol, betaxolol, bevantolol, bisoprolol, bopindolol, bunitrolol, butofilolol, carazolol, carteolol, carvedilol, celiprolol, cetamolol, epanolol, indenolol, mepindolol, metipranolol, metoprolol, moprolol, nadolol, nipradilol, oxprenolol, penbutolol, pindolol, propanolol, talinolol, tertatolol, timolol and toliprolol.

c. Repolarization Prolonging Agents

Non-limiting examples of an agent that prolong repolarization, also known as a Class III antiarrhythmic agent, include amiodarone (cordarone) and sotalol (betapace).

d. Calcium Channel Blockers/Antagonist

Non-limiting examples of a calcium channel blocker, otherwise known as a Class IV antiarrhythmic agent, include an arylalkylamine (e.g., bepridile, diltiazem, fendiline, gallopamil, prenylamine, terodiline, verapamil), a dihydropyridine derivative (felodipine, isradipine, nicardipine, nifedipine, nimodipine, nisoldipine, nitrendipine) a piperazinde derivative (e.g., cinnarizine, flunarizine, lidoflazine) or a micellaneous calcium channel blocker such as bencyclane, etafenone, magnesium, mibefradil or perhexiline. In certain embodiments a calcium channel blocker comprises a long-acting dihydropyridine (amlodipine) calcium antagonist.

e. Miscellaneous Antiarrhythmic Agents

Non-limiting examples of miscellaneous antiarrhymic agents include adenosine (adenocard), digoxin (lanoxin), acecainide, ajmaline, amoproxan, aprindine, bretylium tosylate, bunaftine, butobendine, capobenic acid, cifenline, disopyranide, hydroquinidine, indecainide, ipatropium bromide, lidocaine, lorajmine, lorcainide, meobentine, moricizine, pirmenol, prajmaline, propafenone, pyrinoline, quinidine polygalacturonate, quinidine sulfate and viquidil.

6. Antihypertensive Agents

Non-limiting examples of antihypertensive agents include sympatholytic, alpha/beta blockers, alpha blockers, anti-angiotensin II agents, beta blockers, calcium channel blockers, vasodilators and miscellaneous antihypertensives.

a. Alpha Blockers

Non-limiting examples of an alpha blocker, also known as an α-adrenergic blocker or an α-adrenergic antagonist, include amosulalol, arotinolol, dapiprazole, doxazosin, ergoloid mesylates, fenspiride, indoramin, labetalol, nicergoline, prazosin, terazosin, tolazoline, trimazosin and yohimbine. In certain embodiments, an alpha blocker may comprise a quinazoline derivative. Non-limiting examples of quinazoline derivatives include alfuzosin, bunazosin, doxazosin, prazosin, terazosin and trimazosin.

b. Alpha/Beta Blockers

In certain embodiments, an antihypertensive agent is both an alpha and beta adrenergic antagonist. Non-limiting examples of an alpha/beta blocker comprise labetalol (normodyne, trandate).

c. Anti-Angiotension II Agents

Non-limiting examples of anti-angiotension II agents include include angiotensin converting enzyme inhibitors and angiotension II receptor antagonists. Non-limiting examples of angiotension converting enzyme inhibitors (ACE inhibitors) include alacepril, enalapril (vasotec), captopril, cilazapril, delapril, enalaprilat, fosinopril, lisinopril, moveltopril, perindopril, quinapril and ramipril. Non-limiting examples of an angiotensin II receptor blocker, also known as an angiotension II receptor antagonist, an ANG receptor blocker or an ANG-II type-1 receptor blocker (ARBS), include angiocandesartan, eprosartan, irbesartan, losartan and valsartan.

d. Sympatholytics

Non-limiting examples of a sympatholytic include a centrally acting sympatholytic or a peripherially acting sympatholytic. Non-limiting examples of a centrally acting sympatholytic, also known as a central nervous system (CNS) sympatholytic, include clonidine (catapres), guanabenz (wytensin) guanfacine (tenex) and methyldopa (aldomet). Non-limiting examples of a peripherally acting sympatholytic include a ganglion blocking agent, an adrenergic neuron blocking agent, a β-adrenergic blocking agent or a alpha1-adrenergic blocking agent. Non-limiting examples of a ganglion blocking agent include mecamylamine (inversine) and trimethaphan (arfonad). Non-limiting of an adrenergic neuron blocking agent include guanethidine (ismelin) and reserpine (serpasil). Non-limiting examples of a β-adrenergic blocker include acenitolol (sectral), atenolol (tenormin), betaxolol (kerlone), carteolol (cartrol), labetalol (normodyne, trandate), metoprolol (lopressor), nadanol (corgard), penbutolol (levatol), pindolol (visken), propranolol (inderal) and timolol (blocadren). Non-limiting examples of alpha1-adrenergic blocker include prazosin (minipress), doxazocin (cardura) and terazosin (hytrin).

e. Vasodilators

In certain embodiments a cardiovasculator therapeutic agent may comprise a vasodilator (e.g., a cerebral vasodilator, a coronary vasodilator or a peripheral vasodilator). In certain preferred embodiments, a vasodilator comprises a coronary vasodilator. Non-limiting examples of a coronary vasodilator include amotriphene, bendazol, benfurodil hemisuccinate, benziodarone, chloracizine, chromonar, clobenfurol, clonitrate, dilazep, dipyridamole, droprenilamine, efloxate, erythrityl tetranitrane, etafenone, fendiline, floredil, ganglefene, herestrol bis(b-diethylaminoethyl ether), hexobendine, itramin tosylate, khellin, lidoflanine, mannitol hexanitrane, medibazine, nicorglycerin, pentaerythritol tetranitrate, pentrinitrol, perhexiline, pimethylline, trapidil, tricromyl, trimetazidine, trolnitrate phosphate and visnadine.

In certain aspects, a vasodilator may comprise a chronic therapy vasodilator or a hypertensive emergency vasodilator. Non-limiting examples of a chronic therapy vasodilator include hydralazine (apresoline) and minoxidil (loniten). Non-limiting examples of a hypertensive emergency vasodilator include nitroprusside (nipride), diazoxide (hyperstat IV), hydralazine (apresoline), minoxidil (loniten) and verapamil.

f. Miscellaneous Antihypertensives

Non-limiting examples of miscellaneous antihypertensives include ajmaline, γ-aminobutyric acid, bufeniode, cicletainine, ciclosidomine, a cryptenamine tannate, fenoldopam, flosequinan, ketanserin, mebutamate, mecamylamine, methyldopa, methyl 4-pyridyl ketone thiosemicarbazone, muzolimine, pargyline, pempidine, pinacidil, piperoxan, primaperone, a protoveratrine, raubasine, rescimetol, rilmenidene, saralasin, sodium nitrorusside, ticrynafen, trimethaphan camsylate, tyrosinase and urapidil.

In certain aspects, an antihypertensive may comprise an arylethanolamine derivative, a benzothiadiazine derivative, a N-carboxyalkyl(peptide/lactam) derivative, a dihydropyridine derivative, a guanidine derivative, a hydrazines/phthalazine, an imidazole derivative, a quanternary ammonium compound, a reserpine derivative or a suflonamide derivative.

Arylethanolamine Derivatives. Non-limiting examples of arylethanolamine derivatives include amosulalol, bufuralol, dilevalol, labetalol, pronethalol, sotalol and sulfinalol.

Benzothiadiazine Derivatives. Non-limiting examples of benzothiadiazine derivatives include althizide, bendroflumethiazide, benzthiazide, benzylhydrochlorothiazide, buthiazide, chlorothiazide, chlorthalidone, cyclopenthiazide, cyclothiazide, diazoxide, epithiazide, ethiazide, fenquizone, hydrochlorothizide, hydroflumethizide, methyclothiazide, meticrane, metolazone, paraflutizide, polythizide, tetrachlormethiazide and trichlormethiazide.

N-carboxyalkyl(peptide/lactam) Derivatives. Non-limiting examples of N-carboxyalkyl(peptide/lactam) derivatives include alacepril, captopril, cilazapril, delapril, enalapril, enalaprilat, fosinopril, lisinopril, moveltipril, perindopril, quinapril and ramipril.

Dihydropyridine Derivatives. Non-limiting examples of dihydropyridine derivatives include amlodipine, felodipine, isradipine, nicardipine, nifedipine, nilvadipine, nisoldipine and nitrendipine.

Guanidine Derivatives. Non-limiting examples of guanidine derivatives include bethanidine, debrisoquin, guanabenz, guanacline, guanadrel, guanazodine, guanethidine, guanfacine, guanochlor, guanoxabenz and guanoxan.

Hydrazines/Phthalazines. Non-limiting examples of hydrazines/phthalazines include budralazine, cadralazine, dihydralazine, endralazine, hydracarbazine, hydralazine, pheniprazine, pildralazine and todralazine.

Imidazole Derivatives. Non-limiting examples of imidazole derivatives include clonidine, lofexidine, phentolamine, tiamenidine and tolonidine.

Quanternary Ammonium Compounds. Non-limiting examples of quanternary ammonium compounds include azamethonium bromide, chlorisondamine chloride, hexamethonium, pentacynium bis(methylsulfate), pentamethonium bromide, pentolinium tartrate, phenactropinium chloride and trimethidinium methosulfate.

Reserpine Derivatives. Non-limiting examples of reserpine derivatives include bietaserpine, deserpidine, rescinnamine, reserpine and syrosingopine.

Suflonamide Derivatives. Non-limiting examples of sulfonamide derivatives include ambuside, clopamide, furosemide, indapamide, quinethazone, tripamide and xipamide.

7. Vasopressors

Vasopressors generally are used to increase blood pressure during shock, which may occur during a surgical procedure. Non-limiting examples of a vasopressor, also known as an antihypotensive, include amezinium methyl sulfate, angiotensin amide, dimetofrine, dopamine, etifelmin, etilefrin, gepefrine, metaraminol, midodrine, norepinephrine, pholedrine and synephrine.

8. Treatment Agents for Congestive Heart Failure

Non-limiting examples of agents for the treatment of congestive heart failure include anti-angiotension II agents, afterload-preload reduction treatment, diuretics and inotropic agents.

a. Afterload-Preload Reduction

In certain embodiments, an animal patient that can not tolerate an angiotension antagonist may be treated with a combination therapy. Such therapy may combine adminstration of hydralazine (apresoline) and isosorbide dinitrate (isordil, sorbitrate).

b. Diuretics

Non-limiting examples of a diuretic include a thiazide or benzothiadiazine derivative (e.g., althiazide, bendroflumethazide, benzthiazide, benzylhydrochlorothiazide, buthiazide, chlorothiazide, chlorothiazide, chlorthalidone, cyclopenthiazide, epithiazide, ethiazide, ethiazide, fenquizone, hydrochlorothiazide, hydroflumethiazide, methyclothiazide, meticrane, metolazone, paraflutizide, polythizide, tetrachloromethiazide, trichlormethiazide), an organomercurial (e.g., chlormerodrin, meralluride, mercamphamide, mercaptomerin sodium, mercumallylic acid, mercumatilin dodium, mercurous chloride, mersalyl), a pteridine (e.g., furterene, triamterene), purines (e.g., acefylline, 7-morpholinomethyltheophylline, pamobrom, protheobromine, theobromine), steroids including aldosterone antagonists (e.g., canrenone, oleandrin, spironolactone), a sulfonamide derivative (e.g., acetazolamide, ambuside, azosemide, bumetanide, butazolamide, chloraminophenamide, clofenamide, clopamide, clorexolone, diphenylmethane-4,4′-disulfonamide, disulfamide, ethoxzolamide, furosemide, indapamide, mefruside, methazolamide, piretanide, quinethazone, torasemide, tripamide, xipamide), a uracil (e.g., aminometradine, amisometradine), a potassium sparing antagonist (e.g., amiloride, triamterene) or a miscellaneous diuretic such as aminozine, arbutin, chlorazanil, ethacrynic acid, etozolin, hydracarbazine, isosorbide, mannitol, metochalcone, muzolimine, perhexiline, ticrnafen and urea.

c. Inotropic Agents

Non-limiting examples of a positive inotropic agent, also known as a cardiotonic, include acefylline, an acetyldigitoxin, 2-amino-4-picoline, amrinone, benfurodil hemisuccinate, bucladesine, cerberosine, camphotamide, convallatoxin, cymarin, denopamine, deslanoside, digitalin, digitalis, digitoxin, digoxin, dobutamine, dopamine, dopexamine, enoximone, erythrophleine, fenalcomine, gitalin, gitoxin, glycocyamine, heptaminol, hydrastinine, ibopamine, a lanatoside, metamivam, milrinone, nerifolin, oleandrin, ouabain, oxyfedrine, prenalterol, proscillaridine, resibufogenin, scillaren, scillarenin, strphanthin, sulmazole, theobromine and xamoterol.

In particular aspects, an intropic agent is a cardiac glycoside, a beta-adrenergic agonist or a phosphodiesterase inhibitor. Non-limiting examples of a cardiac glycoside includes digoxin (lanoxin) and digitoxin (crystodigin). Non-limiting examples of a β-adrenergic agonist include albuterol, bambuterol, bitolterol, carbuterol, clenbuterol, clorprenaline, denopamine, dioxethedrine, dobutamine (dobutrex), dopamine (intropin), dopexamine, ephedrine, etafedrine, ethylnorepinephrine, fenoterol, formoterol, hexoprenaline, ibopamine, isoetharine, isoproterenol, mabuterol, metaproterenol, methoxyphenamine, oxyfedrine, pirbuterol, procaterol, protokylol, reproterol, rimiterol, ritodrine, soterenol, terbutaline, tretoquinol, tulobuterol and xamoterol. Non-limiting examples of a phosphodiesterase inhibitor include amrinone (inocor).

d. Antianginal Agents

Antianginal agents may comprise organonitrates, calcium channel blockers, beta blockers and combinations thereof. Non-limiting examples of organonitrates, also known as nitrovasodilators, include nitroglycerin (nitro-bid, nitrostat), isosorbide dinitrate (isordil, sorbitrate) and amyl nitrate (aspirol, vaporole).

### J. Surgical Therapeutic Agents

In certain aspects, the secondary therapeutic agent may comprise a surgery of some type, which includes, for example, preventative, diagnostic or staging, curative and palliative surgery. Surgery, and in particular a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

### K. Drug Formulations and Routes for Administration to Patients

It will be understood that in the discussion of formulations and methods of treatment, references to any compounds are meant to also include the pharmaceutically acceptable salts, as well as pharmaceutical compositions. Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector or cells, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present invention, its use in therapeutic compositions is contemplated. Supplementary active ingredients also can be incorporated into the compositions, provided they do not inactivate the vectors or cells of the compositions.

In specific embodiments of the invention the pharmaceutical formulation will be formulated for delivery via rapid release, other embodiments contemplated include but are not limited to timed release, delayed release, and sustained release. Formulations can be an oral suspension in either the solid or liquid form. In further embodiments, it is contemplated that the formulation can be prepared for delivery via parenteral delivery, or used as a suppository, or be formulated for subcutaneous, intravenous, intramuscular, intraperitoneal, sublingual, transdermal, or nasopharyngeal delivery.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients, which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the technique described in the U.S. Pat. Nos. 4,256,108; 4,166,452; and 4,265,874 to form osmotic therapeutic tablets for controlled release (hereinafter incorporated by reference).

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain an active material in a mixture with excipients suitable for the manufacture of aqueous suspensions. Such excipients are suspending agents, for example sodium carboxymethylcellulose, methylcellulose, hydroxy-propylmethycellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose, saccharin or aspartame.

Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

Pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavouring agents.

Syrups and elixirs may be formulated with sweetening agents, for example glycerol, propylene glycol, sorbitol or sucrose. Such formulations may also contain a demulcent, a preservative and flavoring and coloring agents. Pharmaceutical compositions may be in the form of a sterile injectable aqueous or oleagenous suspension. Suspensions may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

Compounds may also be administered in the form of suppositories for rectal administration of the drug. These compositions can be prepared by mixing a therapeutic agent with a suitable non-irritating excipient which is solid at ordinary temperatures, but liquid at the rectal temperature and will therefore melt in the rectum to release the drug. Such materials are cocoa butter and polyethylene glycols.

For topical use, creams, ointments, jellies, gels, epidermal solutions or suspensions, etc., containing a therapeutic compound are employed. For purposes of this application, topical application shall include mouthwashes and gargles.

Formulations may also be administered as nanoparticles, liposomes, granules, inhalants, nasal solutions, or intravenous admixtures

The previously mentioned formulations are all contemplated for treating patients suffering from heart failure or hypertrophy.

The amount of active ingredient in any formulation may vary to produce a dosage form that will depend on the particular treatment and mode of administration. It is further understood that specific dosing for a patient will depend upon a variety of factors including age, body weight, general health, sex, diet, time of administration, route of administration, rate of excretion, drug combination and the severity of the particular disease undergoing therapy.

### V. Screening Methods

The present invention further comprises methods for identifying inhibitors of Ku or broadly inhibitors of cardiac hypertrophy, heart failure, or cardiovascular disease. These assays may be performed in cells and may be useful in identifying compounds or agents indicated for the prevention or treatment or reversal of cardiac hypertrophy or heart failure. These assays may comprise random screening of large libraries of candidate substances; alternatively, the assays may be used to focus on particular classes of compounds selected with an eye towards structural attributes that are believed to make them more likely to inhibit the function or activity or expression or stability of Ku.

In one embodiment for identifying an inhibitor of Ku, one will determine the expression of α-MyHC promoter driven reporter gene in the presence and absence of the candidate substance. For example, a method generally comprises:
(a) providing a cell that has an intact αMyHC promoter operably linked to a reporter gene, wherein said cell expresses sufficient levels of Ku70 and Ku80 to operably repress the α-MyHC promoter;
(b) contacting said cell with a candidate inhibitor substance; and
(c) monitoring the cell for an increase in expression of the reporter gene in the presence of said candidate Ku inhibitor as compared to the expression of a cell in the absence of said candidate inhibitor;
   wherein an increase in expression of the reporter gene in the cell, as compared to an untreated cell, identifies the candidate substance as an inhibitor of Ku as well as a potential inhibitor of heart failure or cardiac hypertrophy (increased expression again may be increased RNA or protein expression, and it may be due to an indirect effect on another gene or gene product causing the increased expression of the reporter).

Assays also may be conducted in isolated cells, organs, or in living organisms.

It will, of course, be understood that all the screening methods of the present invention are useful in themselves notwithstanding the fact that effective candidates may not be found. The invention provides methods for screening for such candidates, not solely methods of finding them.

### A. Modulators

As used herein the term “candidate substance” refers to any molecule that may potentially inhibit the activity or cellular functions of Ku, including those that block Ku expression. The candidate substance may be a protein or fragment thereof, a small molecule, or even a nucleic acid. It may prove to be the case that the most useful pharmacological compounds will be compounds that are discovered through high-throughput screens of large compound libraries. Using lead compounds to help develop improved compounds is known as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration, or which may affect the function of various other molecules. In one approach, one would generate a three-dimensional structure for a target molecule, or a fragment thereof. This could be accomplished by x-ray crystallography, computer modeling, or by a combination of both approaches.

It also is possible to use antibodies to ascertain the structure of a target compound, activator, or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecular libraries that are believed to meet the basic criteria for useful drugs in an effort to “brute force” the identification of useful compounds. Screening of such libraries, including combinatorially-generated libraries (e.g., peptide libraries), is a rapid and efficient way to screen large number of related (and unrelated) compounds for activity. Combinatorial approaches also lend themselves to rapid evolution of potential drugs by the creation of second, third, and fourth generation compounds modeled on active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for Ku. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

### B. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads. Such assays are most directly applicable in the identification of molecules that bind to Ku, thereby reducing or eliminating its function.

A technique for high throughput screening of compounds is described in WO 84/03564. Large numbers of small peptide test compounds are synthesized on a solid substrate, such as plastic pins or some other surface. Such peptides could be rapidly screened for their ability to inhibit Ku function.

### C. In Cyto Assays

The present invention also contemplates the screening of compounds for their ability to inhibit Ku activity in cells. Various cell lines can be utilized for such screening assays, including cells specifically engineered for this purpose.

### D. In Vivo Assays

In vivo assays involve the use of various animal models of heart disease, including transgenic animals, that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

Treatment of animals with test compounds will involve the administration of the compound, in an appropriate form, to the animal. Administration will be by any route that could be utilized for clinical purposes. Determining the effectiveness of a compound in vivo may involve a variety of different criteria, including but not limited to toxicity measurements, efficacy measurements, bioavailability and drug half-life measurements. Also, measuring toxicity and dose response can be performed in animals in a more meaningful fashion than in in vitro or in cyto assays.

### VI. Vectors for Cloning, Gene Transfer and Expression

Within certain embodiments, expression vectors are employed to express various products including Ku and α-MyHC, antisense molecules, ribozymes or interfering RNAs. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

### A. Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In certain embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, the native α-MyHC promoter will be employed to drive expression of the corresponding gene, a heterologous gene, a screenable or selectable marker gene, or any other gene of interest.

In other embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 1 and 2 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 1 and Table 2). Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 1
Promoter and/or Enhancer

| Promoter/Enhancer | References |
|---|---|
| Immunoglobulin Heavy Chain | Banerji et al., 1983; Gilles et al., 1983; Grossched1 |
| | et al., 1985; Atchinson et al., 1986, 1987; Imler et |
| | al., 1987; Weinberger et al., 1984; Kiledjian et al., |
| | 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo et |
| | al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et |
| | al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al., 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi et |
| | al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 2
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa et |
| | | al., 1987, Karin et al., |
| | | 1987; Angel et al., 1987b; |
| | | McNeall et al., 1989 |
| MMTV (mouse | Glucocorticoids | Huang et al., 1981; Lee et |
| mammary tumor | | al., 1981; Majors et al., |
| virus) | | 1983; Chandler et al., |
| | | 1983; Ponta et al., 1985; |
| | | Sakai et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Of particular interest are muscle specific promoters, and more particularly, cardiac specific promoters. These include the myosin light chain-2 promoter (Franz et al., 1994; Kelly et al., 1995), the alpha actin promoter (Moss et al., 1996), the troponin 1 promoter (Bhavsar et al., 1996); the Na⁺/Ca²⁺ exchanger promoter (Barnes et al., 1997), the dystrophin promoter (Kimura et al., 1997), the alpha7 integrin promoter (Ziober & Kramer, 1996), the brain natriuretic peptide promoter (LaPoint et al., 1995) the α-B-crystallin/small heat shock protein promoter (Gopal-Srivastava, R., 1995), and the ANF promoter (LaPointe et al., 1988).

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

### B. Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

### C. Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

### D. Delivery of Expression Vectors

There are a number of ways in which expression vectors may be introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986). These have a relatively low capacity for foreign DNA sequences and have a restricted host spectrum. Furthermore, their oncogenic potential and cytopathic effects in permissive cells raise safety concerns. They can accommodate only up to 8 kB of foreign genetic material but can be readily introduced in a variety of cell lines and laboratory animals (Nicolas and Rubenstein, 1988; Temin, 1986).

One of the preferred methods for in vivo delivery involves the use of an adenovirus expression vector. “Adenovirus expression vector” is meant to include those constructs containing adenovirus sequences sufficient to (a) support packaging of the construct and (b) to express an antisense polynucleotide that has been cloned therein. In this context, expression does not require that the gene product be synthesized.

The expression vector comprises a genetically engineered form of adenovirus. Knowledge of the genetic organization of adenovirus, a 36 kB, linear, double-stranded DNA virus, allows substitution of large pieces of adenoviral DNA with foreign sequences up to 7 kB (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP—located at 16.8 m.u.—is particularly efficient during the late phase of infection, and all the mRNAs issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in the E1, the D3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of the current adenovirus vector is under 7.5 kb, or about 15% of the total length of the vector. More than 80% of the adenovirus viral genome remains in the vector backbone and is the source of vector-borne cytotoxicity. Also, the replication deficiency of the E1-deleted virus is incomplete.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors, as described by Karlsson et al. (1986), or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1991). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al (1991) introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after.

In order to effect expression of sense or antisense gene constructs, the expression construct must be delivered into a cell. This delivery may be accomplished in vitro, as in laboratory procedures for transforming cells lines, or in vivo or ex vivo, as in the treatment of certain disease states. One mechanism for delivery is via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, E.P. App. 273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

### VII. Preparing Antibodies to Ku

In yet another aspect, the present invention contemplates the use of antibodies that may bind to Ku or some associated factor or protein involved in the disease process mediated by Ku. An antibody can be a polyclonal or a monoclonal antibody, it can be humanized, single chain, or even an Fab fragment. In a preferred embodiment, an antibody is a monoclonal antibody. Means for preparing and characterizing antibodies are well known in the art (see Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to Ku antigen epitopes.

In general, both polyclonal, monoclonal, and single-chain antibodies against Ku may be used in a variety of embodiments. A particularly useful application of such antibodies is in purifying native or recombinant Ku, for example, using an antibody affinity column. The operation of all accepted immunological techniques will be known to those of skill in the art in light of the present disclosure.

Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified PKD protein, polypeptide or peptide or cell expressing high levels of PKD. The immunizing composition is administered in a manner effective to stimulate antibody producing cells. Rodents such as mice and rats are preferred animals, however, the use of rabbit, sheep or frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

### VIII. Definitions

As used herein, the term “heart failure” is broadly used to mean any condition that reduces the ability of the heart to pump blood. As a result, congestion and edema develop in the tissues. Most frequently, heart failure is caused by decreased contractility of the myocardium, resulting from reduced coronary blood flow; however, many other factors may result in heart failure, including damage to the heart valves, vitamin deficiency, and primary cardiac muscle disease. Though the precise physiological mechanisms of heart failure are not entirely understood, heart failure is generally believed to involve disorders in several cardiac autonomic properties, including sympathetic, parasympathetic, and baroreceptor responses. The phrase “manifestations of heart failure” is used broadly to encompass all of the sequelae associated with heart failure, such as shortness of breath, pitting edema, an enlarged tender liver, engorged neck veins, pulmonary rales and the like including laboratory findings associated with heart failure.

The term “treatment” or grammatical equivalents encompasses the prevention, improvement and/or reversal of symptoms of a specific disease, disorder, syndrome or state (i.e., improving the ability of the heart to pump blood in a heart failure setting). Improvement in the physiologic function of the heart may be assessed using any of the measurements described herein (e.g., measurement of ejection fraction, fractional shortening, left ventricular internal dimension, heart rate, etc.), as well as any effect upon the animal's survival. A compound which causes an improvement in any parameter associated with a specific disease used in the screening methods of the instant invention may thereby be identified as a therapeutic compound.

The terms “compound” and “chemical agent” may refer to any chemical entity, pharmaceutical, drug, protein, antibody, nucleic acid and the like that can be used to treat or prevent a disease, illness, sickness, or disorder of bodily function. Compounds and chemical agents comprise both known and potential therapeutic compounds. A compound or chemical agent can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of heart failure.

As used herein, the term “cardiac hypertrophy” refers to the process in which adult cardiac myocytes respond to a wide variety of pathophysiological, chemical, external and biological stresses through hypertrophic growth. Such growth is characterized by cell size increases without cell division, assembling of additional sarcomeres within the cell to maximize force generation, and an activation of a fetal cardiac gene program. Cardiac hypertrophy is often associated with increased risk of morbidity and mortality, and thus studies aimed at understanding the molecular mechanisms of cardiac hypertrophy could have a significant impact on human health.

As used herein, the term “inhibitor” refers to any agent which is capable of decreasing the expression, stability, activity, or potency of Ku. Inhibitors may include proteins, nucleic acids, carbohydrates, peptides, small molecules, antibodies, or any other molecule(s) which binds or interacts with a receptor, molecule, and/or pathway of interest. Inhibitors need not act directly on Ku protein or the gene locus, but may cause a downregulation of expression or activity or function (at the RNA or protein level) indirectly, via an effect on some other gene or protein that leads to downregulation or decreased expression of Ku.

As used herein, the term “modulate” refers to a change or an alteration in a biological or chemical activity. Modulation may be an increase or a decrease in protein activity, a change in kinase activity, a change in binding characteristics, or any other change in the biological, functional, or immunological properties associated with the activity of a protein or other structure of interest. The term “modulator” refers to any molecule or compound which is capable of changing or altering biological activity as described above.

As used herein, the term “select” or “selection” in the context of an inhibitor will be understood to mean making a choice between known or experimental compounds and agents.

As used herein, the term “small molecule” refers to an organic molecule or its salt(s), usually having a molecular weight less than 1000 Daltons.

### IX. EXAMPLES

The following examples are included to further illustrate various aspects of the invention. It should be appreciated by those of skill in the art that the techniques disclosed in the examples which follow represent techniques and/or compositions discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1

### Materials and Methods

Antibodies: YY1 (SC-7341×) and Ku80 (SC-5280) antibodies were purchased from SantaCruz Biotech. Ku70 antibody (NB 100-102) was purchased from Novus Biological. For immunoprecipitation experiments, the YY1 and Ku antibodies were purchased from Santa Cruz Biotech. The alkaline phosphatase (115-053-146), HRP (115-035-146) anti-mouse and HRP (711-035-152) anti-rabbit were purchased from Jackson Laboratories.

Plasmid construct: The −454/+32 bp fragment of the human α-MyHC promoter was cloned into the pGL3 basic vector (Promega). Mutations were created in the Ku binding site by generating oligonucleotides (see EMSA) containing the mutation of interest and amplifying the fragments containing the mutation by PCR. The YY1 expression construct was a gift from Dr. Michael Atchison (Univ. of Pennsylvania) and the Ku expression construct was a gift from Dr. William Dynan (Medical College of Georgia). The DNA constructs were purified using the Qiagen method.

Cell Culture and Transfection: Neonatal rat ventricular myocytes (NRVM) were prepared according to the method described in Waspe et al. (1990). Briefly, 150,000 cells/well were plated in 12-well tissue culture plates coated with gelatin. Eighteen hours later, the media was changed to MEM supplemented with Hank's salt and L-glutamine. 20 mM Hepes pH 7.5, Penicillin, Vitamin B12, BSA, insulin and transferin were added to the media. Transfections were carried out by the Fugene 6 (Roche) method according to manufacturer's recommendations; 0.75 μl of Fugene/0.25 μg of plasmid DNA were transfected in each well. In the co-transfection experiments, the total amount of DNA was kept constant by the addition of a plasmid containing the CMV promoter not driving the expression of any gene.

Preparation of protein extracts: Protein extracts from human normal and failing (IDCidiopathic dilated cardiomyopathy) left ventricles were prepared according to Molkentin and Markham (1993) with minor modifications. 0.5 g of tissue was homogeneized with a teflon homogenizer attached to a drill (SKIL—PA6-GF30) at 50% power. The resulting preparation was sonicated in a cell disruptor (Ultrasonics—W185F) at 50% power for 15 seconds. Following cell lysis, the proteins were precipitated by the slow addition of an equal volume of 4M NH₄SO₄. These results in a 50% final concentration of NH4SO4, allowing a greater protein recovery when compared to the 30% final concentration of NH4SO4 as described in the original method. HeLa cell nuclear extracts were prepared by the method of Dignam et al. (1983).

Eletrophoretic Mobility Shift Assays (EMSAs): were carried out as described (Sucharov et al, 1995). Thirty base pairs double-stranded oligonucleotides were labeled by Klenow fill-in using ³²P dCTP. The reaction was performed using 100,000 cpm of wild-type or mutant probe in a 30 μl binding reaction containing 10 mM Hepes pH7.9, 100 mM KCL, 4% glycerol, 1 mM EDTA, 0.1% Nonidet P-40, 1 μg poly dI-dC (Pharmacia Biotech) and 10 μg of extract. The resulting complex was resolved in a non-denaturing 4% acrylamide gel in 0.5×TBE.

EMSA using circular DNA was done essentially as described (Giffin et al., 1996). An oligonucleotide containing three copies of the above fragment was cloned into the mini circle essentially as described (Giffin et al., 1996).

Purification of Ku70/Ku80: HeLa cell pellets were bought from Computer Cell Culture Center, Belgium and nuclear extract was prepared as described above. The double-stranded (ds) oligonucleotide used in these experiments had the YY1 binding site mutated. A multimerized oligonucleotide containing three copies of the Ku binding site was labeled with biotin on the 5′ end. 20 mg of dynabeads (Dynal) coupled to 3 nmoles of the ds oligonucleotide were washed three times in binding buffer (10 mM Hepes, pH 7.9, 4% glycerol, 1 mM EDTA, 0.1% Nonidet P-40, and 200 μg/ml insulin, supplemented with 1× protease inhibitors (Boehringer-Mannheim). 1 mg of HeLa cells nuclear extract was incubated with the oligonucleotide-coupled magnetic beads for 30 minutes at room temperature. The beads containing bound proteins were washed three times, 5 minutes each, with 500 μl of binding buffer containing 100 mM KCL. For the last two washes, 15 μg of poly dI-dC was added. Proteins were step-eluted using 100 μl of binding buffer containing 0.2, 0.3 or 1M KCl, for 10 min each. 1 μl of each fraction and 5 μl of each wash were assayed by EMSA, without poly dI-dC. The peak fraction (1M) of the purification was submitted to denaturing polyacrylamide gel electrophoresis and stained with colloidal Coomassie (Invitrogen) and destained with water. Images of the gels were captured with a Umax scanner.

In-gel Tryptic Protein Digest and Sample Preparation for Mass Spectrometry: Protein bands were excised from the gels with a scalpel. Gel pieces were washed 2× for 10 min with 200 μl 50% acetonitrile (CH₃CN)/25 mM ammonium bicarbonate (NH₄HCO₃) and then once with 200 μl 100% CH₃CN. The gel pieces were dried in a Speed-vac for 15 min. To the dried gel pieces was added 20 μl of 20 μg/ml trypsin (Promega sequencing grade) in 50 mM NH₄HCO₃. The gel pieces were allowed to rehydrate on ice for 20 min. and incubated overnight at 37° C. The next day the peptides were extracted into 200 μl of 50% CH₃CN/0.1% trifluoroacetic acid (TFA) with vigorous mixing.

The extracts were transferred to fresh tubes and taken to dryness (Speedvac). The peptides were then redissolved overnight in 20 μl of 0.1% TFA. ZipTips (C18, 0.6 μl bed volume, Millipore) were wetted with 50% CH₃CN/0.1% TFA and equilibrated in 0.1% TFA. Peptides were bound by pipetting 10× through the bed. The ZipTips were then washed 3× with 0.1% TFA and the last wash was completely expelled. For each sample a 2 μl aliquot of 80% CH₃CN/0.1% TFA was placed on a stainless steel MALDI-TOF (matrix assisted laser desorption/ionization-time of flight) plate. The ZipTip was brought into contact with this solution which was pipetted 5× through the bed to elute the peptides. Immediately a 1 μl aliquot of matrix solution was spotted on top of the eluate and allowed to dry. The matrix solution consisted of 10 mg/ml recrystallized α-cyano-4-hydroxycinnamic acid dissolved in 80% CH3CN/0.1% TFA. A standard mix of des-Argl bradykinin, angiotensin I, glul-fibrino-peptide B, and ACTH (18-39) in matrix was also applied to an adjacent region of the plate. MALDI-TOF spectra were acquired on a Voyager-DE PRO (PerSeptive Biosystems) instrument operating in reflector mode. Following initial data collection using external calibration on the standard mix, the spectra of unknowns were recalibrated using autolytic tryptic fragments as internal mass markers. Mono-isotopic peptide masses with a signal to noise of greater than 5:1 were entered into the Mascot program (www.matrixscience.com). This is a peptide mass fingerprinting program that finds the best fit of observed tryptic peptides to predicted tryptic peptides from database proteins. These masses were searched at a tolerance of 50 ppm against the NCBInr database. One missed cleavage was allowed. Oxidation of methionine was allowed as a variable modification because this has been observed after colloidal Coomassie staining.

Western Blots: Western Blots were performed essentially as described (Sucharov et al., 2003). Ku antibody was diluted 1:1000 in 1×PBS containing 3% BSA, 2% Normal Goat Serum and 0.1% Tween and incubated with the blot for 1 hour at room temperature. The mouse secondary antibody conjugated to alkaline phosphatase was diluted 1:2500 in 1×PBS containing 5% low fat dry milk and 0.1% tween and incubated with the blot for 1 hour at room temperature. Alkaline phosphataseconjugated secondary antibodies were used along with the Vistra (Amersham) enhanced chemoluminence reagent, an ABI STORM phosphoimager, and ImageQuant Software to visualize and quantify bound antibody.

Immunoprecipitation/immunobloting: Immunoprecipitation experiments were done using YY1, Ku70, Ku80, HA-probe and c-Myc Ab. Experiments were done according to Santa Cruz Biotech recommendations with minor modifications. After 4 washes with 1XRIPA buffer, the sample was incubated with 2-3× packed volume with 2× sample buffer (Bio-Rad) and incubated at room temperature for 30 min. β-mercaptanol was added to the suppernatant after centrifugation and samples were loaded without boiling. Ethidium bromide was added to HeLa nuclear extracts as described (Lai and Herr, 1992) and immunoprecipitation experiments were performed as described above.

Western experiments were done using the above Ab and the anti-mouse or anti-rabbit HRP was used for detection.

Coomassie staining of PVDF membranes: This technique was performed essentially as described (Sucharov et al., 2003). Following Western blot experiments, the membrane was stripped of the antibodies and stained with coomassie for 1 hr. The membrane was than de-stained for at least 12 hrs. The proteins on the membrane were quantified on the LiCor scanner.

Ku70/80 recombinant adenovirus: Methods for generating recombinant adenovirus expression vectors have been described previously (Albert et al., 1995; Schaack and Guo, 1995). The Ku70 and Ku80 adenovirus expression vectors, AdCMV-Ku70 and AdCMV-Ku80, are based on the Cre-dependent luciferase expression vector, AdCUL (Langer et al., 2002). AdCUL consists of oppositely oriented mutant lox sites, lox71 and lox66, flanking an anti-sense firefly luciferase reporter gene downstream of the cytomegalovirus immediate early promoter (CMV). Cre-mediated recombination between lox71 and lox66 inverts the floxed cassette into the sense orientation, resulting in luciferase gene expression (Albert et al., 1995).

Substitution of the Ku70 and Ku80 cDNAs for the luciferase gene in AdCUL generates the antisense expression vectors, AdCMV-α-Ku70 and AdCMV-α-Ku80, respectively. AdCMV-α-Ku70 and AdCMV-α-Ku80 were subsequently passaged through the Cre-expressing cell line HEK293Cre57 (Langer et al., 2002) to facilitate generation of the sense-oriented expression vectors, AdCMV-Ku70 and AdCMVKu80. The emergent virus was plaque purified, and the sense-orientation of the floxed cassettes verified by PCR.

RNase protection assay: Total RNA was extracted by TRIZol (Invitrogen) and used in RNase protection assays (RPA). RPAs were performed essentially as described (Kinugawa et al., 2001; Patten et al., 1996). Briefly, 5 μg of total RNA was hybridized against probes specific to skeletal α-actin, SERCA2a, α-MyHC, β-MyHC and GAPDH. RNase protection experiments were performed using the RPAII kit (Ambion).

Cell Culture and Infection With Antisense Virus: Neonatal rat ventricular myocytes (NRVM) were prepared according to the method described in Waspe et al. (1990). Briefly, 150,000 cells/well were plated in 12-well tissue culture plates coated with gelatin. Eighteen hours later, the media was changed to MEM supplemented with Hank's salt and L-glutamine. 20 mM Hepes pH 7.5, Penicillin, Vitamin B12, BSA, insulin and transferin were added to the media. Infection with anti-sense KU70 or control adenovirus was done at a MOI of 10 pfu/cell.

### B. Results

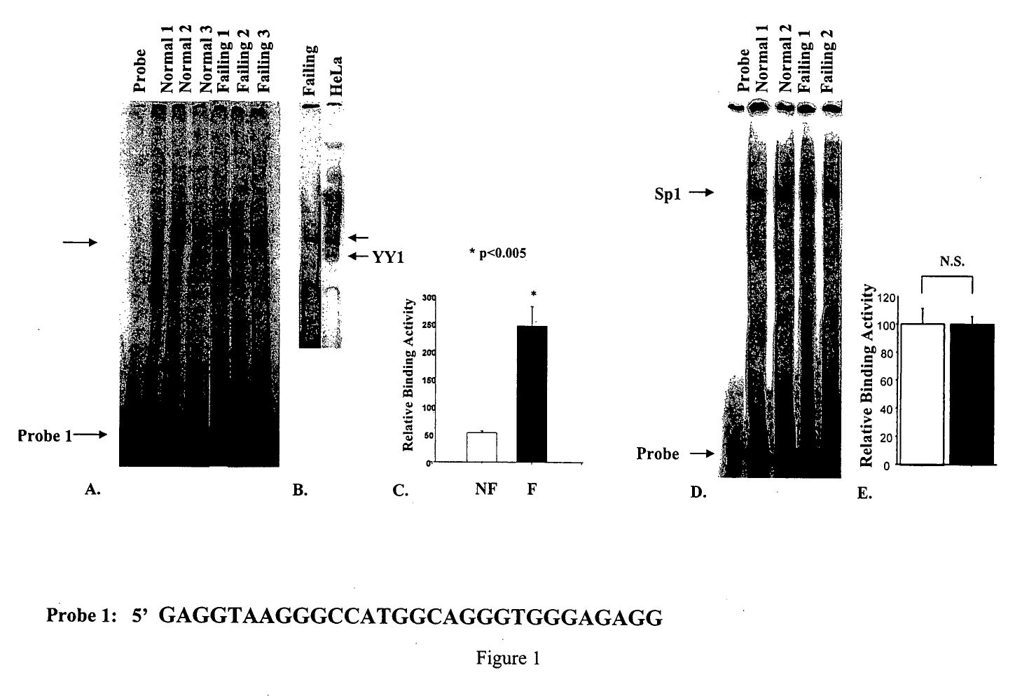

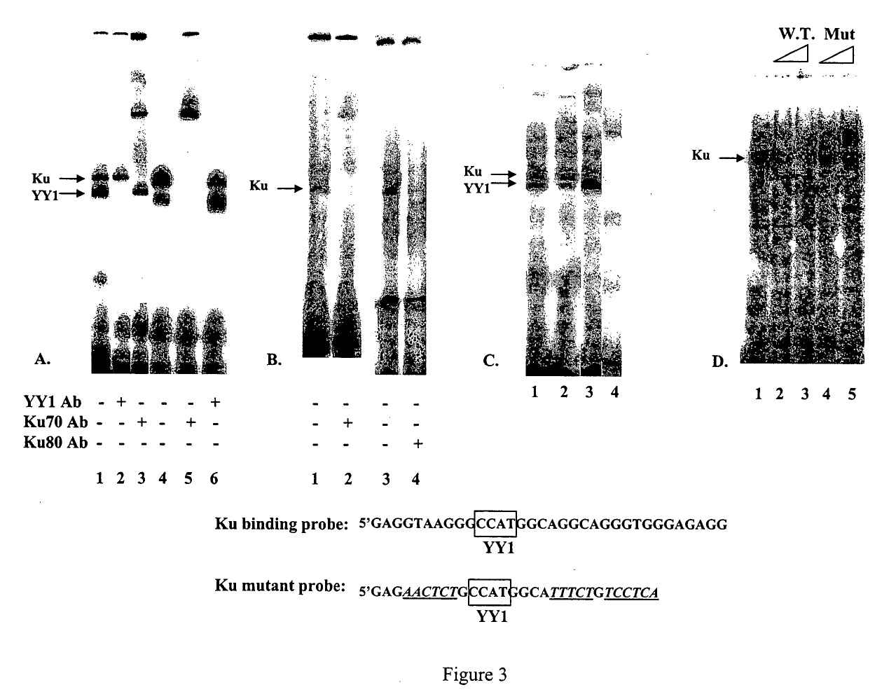

Elevated DNA binding activity to the human α-MyHC promoter in failing human hearts. In an attempt to identify transcription factors that could be responsible for the down regulation of the α-MyHC promoter, several regions of the promoter were chosen for further study based on deletion/promoter activity studies (data not shown) and used as probes in EMSAs against normal and failing human heart extracts. Some of these regions contained consensus binding sites for known transcriptional activators (e.g., GATA4, MEF2, TEF-1). One site (see FIGS. 1A-D) was chosen for further studies because its deletion resulted in a 2-fold increase in α-MyHC promoter activity in NRVMs. This region contains a putative binding site for the YY1 transcription factor which the inventors have shown to function as a repressor (see below; Sucharov et al., 2003). As shown in FIG. 1A, this region also binds a factor which is distinct from YY1 with 5-fold more DNA binding activity in extracts from failing human hearts. As a control, a probe containing a Sp1 binding site was used in EMSA against the same extracts described in FIGS. 1A-D. As seen in FIG. 1D, there is no difference on Sp1 binding activity between failing and normal human hearts, consistent with the specificity of the increase in binding activity being specific. The fact that YY1 cannot be detected binding to this site from failing heart extracts is not understood at the present time; particularly since HeLa cell nuclear extracts form two complexes with this DNA sequence; one that co-migrates with the complex seen in human failing hearts and another faster migrating complex (FIG. 1B). The faster migrating complex contains YY1 as shown by the ability of an anti YY1 antibody to block its formation (FIG. 3A).

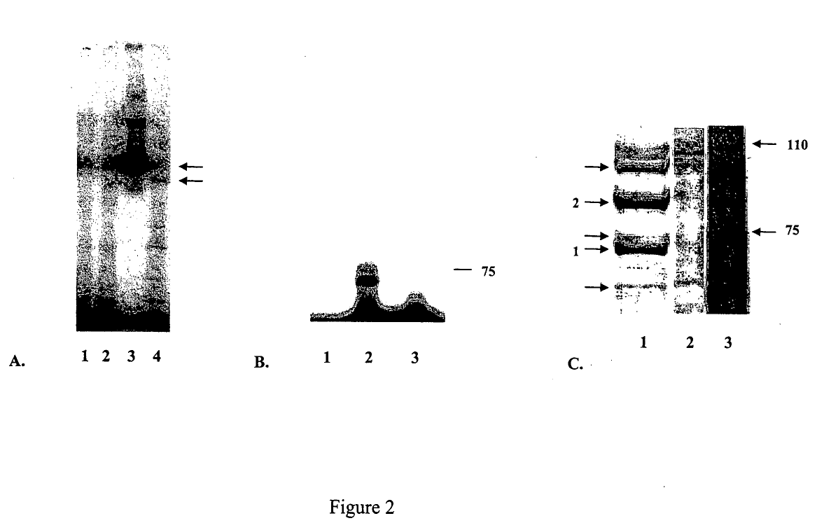

Ku is identified as the protein that binds to the human αMyHC promoter. In order to identify the slower migrating proteins present in the complex from failing human heart and HeLa cells, the oligonucleotide containing the binding site for this complex but with the YY1 site mutated was multimerized, labeled with biotin and incubated with HeLa cell nuclear extracts. The complex was then bound to Dynabeads (Dynal). The eluted fractions (see Methods) were analyzed by EMSA. As seen in FIG. 2A, lane 3, the 1M elution fraction contains an enriched protein with similar migration to the slower migrating complex in HeLa cell nuclear extract (FIG. 2A, lane 4). U.V. cross-linking experiments suggest that the molecular weight of the protein of interest is approximately 70 KDa (FIG. 2B, lane 2). The 1M elution fraction was resolved on a denaturing polyacrylamide gel. Five major bands were obtained (FIG. 2C, lane 1) and all of them were analyzed by mass spectrometry. Of the 5 bands, two bands that showed the greatest intensities (FIG. 2C, bands 1 and 2) were identified as Ku 70 (band 1) with 15 out of 21 peptides matching, 23% sequence coverage, and a Mascot probability based MOWSE score of 187, and Ku 80 (band 2) with 18 out of 24 peptides matching, 17% sequence coverage, and a Mascot probability based MOWSE score of 186. The molecular weights of the bands (70 and 85 KDa) correspond to the molecular weight of Ku70 and Ku80 respectively. The bands are of similar intensity which is consistent with the known hetero-dimerization of these proteins.

Ku is part of the complex that is increased in failing human hearts. Since Ku was identified as binding to the α-MyHC promoter in by mass spectrometry, an antibody against Ku was used in EMSA experiments to determine if Ku is in fact part of the complex that binds to this sequence in HeLa cells and is increased in failing heart extracts. As seen in FIG. 3A, addition of the Ku 70 antibody supershifts the slower migrating complex in a HeLa cell nuclear extract (lane 3). Addition of the YY1 antibody prevents YY1 from binding to the DNA but has no effect on Ku binding (lane 2). Similar results were obtained with purified Ku proteins (lanes 4-6). Interestingly, the addition of purified Ku proteins to the binding experiment generates two complexes. Even though the migration of the faster complex is similar to YY1, it does not contain YY1 since it is not supershifted by the YY1 antibody (lane 6). It seems likely that this band corresponds to Ku70 only since it is supershifted by the Ku70 antibody and the slower migrating band contains Ku70 and Ku80. Addition of Ku70 or Ku80 antibodies also supershifts the complex in binding experiments with human failing heart extracts (FIG. 3B) while the YY1 antibody failed to supershift the complex (data not shown). To determine the sequences in the α-MyHC promoter to which Ku is binding, a series of mutants was created. Since Ku binding sites are quite variable, different mutations were designed. Sequence specific binding of Ku to DNA has been documented in the context of the long terminal repeat (LTR) of the mouse mammary tumor virus (MMTV) (Giffin et al., 1999).

Ku has been shown to bind a sequence containing a direct repeat whose monomer is GAGAAAGA. However, Ku is capable of interacting with a truncated version of this sequence that contains only the monomer. Transversion mutation of the 5th nucleotide to a T in the first repeat prevents binding of Ku (Giffin et al., 1999). Ku has also been shown to bind the sequence GAGAGGGGTCGG (SEQ ID NO:1) in the human CD34 promoter (Taranenko and Krause, 2000). The human α-MyHC promoter contains 2 potential Ku binding sites. The first potential Ku binding site contains a sequence similar to the CD34 promoter and to the MMTV LTR: GAGAGG. The second potential Ku binding site in the human α-MyHC promoter is GTAAG followed by two purines. Camara-Clayette et al., (1999) have proposed a consensus site for Ku that is SHBAGAYAS (S is for G or C; H for A, T or C; B for T, C or G; and Y for C or T). Although not all Ku binding promoters contain this binding site, many of the promoters contain at least 5 of the 9 nucleotides proposed in the consensus binding site (Giffin et al., 1999; Taranenko and Krause, 2000; Camara-Clayette et al., 1999; Kim et al., 1995; Merante et al., 2002; Willis et al., 2002). Both of the potential Ku binding sites in the human α-MyHC promoter region fall into this category. Therefore, mutations were created in both regions. As seen in FIG. 3C, mutations in the two potential Ku binding sites completely abolish binding of Ku but maintain binding of YY1 (lane 3) and mutations in the YY1 site prevent binding of YY1 but allow binding of Ku (lane 2). Probes containing mutations in each of the Ku binding sites were created and used in EMSA. Ku retains the ability to bind to the DNA if either one of the binding sites is present (data not shown). In order to test binding specificity, the YY1 mutant probe was used in EMSA experiments and either the same oligonucleotide or the Ku doubly mutant oligonucleotide was used as cold competitors. As seen in FIG. 3D, the wild-type oligonucleotides can compete for binding to Ku (lanes 2 & 3) while the mutant competitor cannot (lanes 4 & 5). Competition with higher amounts of Ku leads to the appearance of a slower migrating complex. This complex in only seen in these conditions and its identity is not known. These results suggest that the binding of Ku to the αMyHC promoter is sequence-specific.

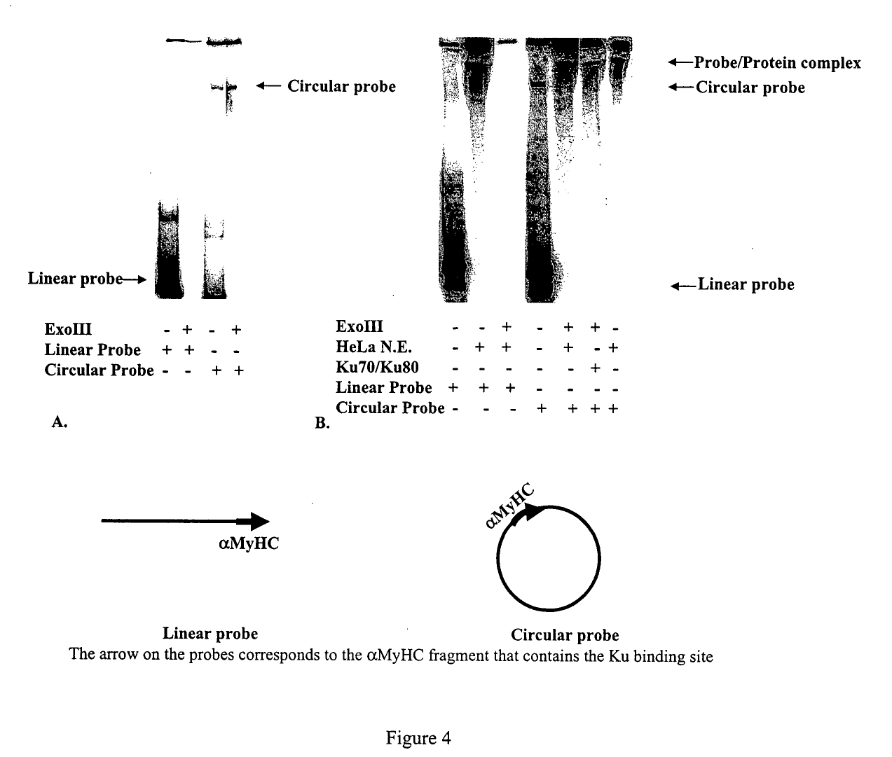

Binding of Ku to the human αMyHC promoter occurs in the absence of DNA ends. The Ku70/80 complex has been shown to bind DNA ends and to be involved in DNA repair (for review see Tuteja and Tuteja, 2002). The complex has also been shown to bind to DNA in a sequence specific manner and function as a transcription factor (for review see Dynan and Yoo, 1998). In order to determine whether the Ku70/80 complex recognizes the human α-MyHC promoter independently of the presence of DNA ends, an oligonucleotide containing three copies of the Ku binding region was labeled and cloned in a mini circle (see methods). As seen in FIGS. 4A-B, Ku is capable of binding the human α-MyHC promoter even when the DNA is in a circular form. FIG. 4A shows that Exo III digests the linear probe to completion but does not affect the circular probe. FIG. 4B shows that the linear probe is capable of binding to Ku70/80, but that upon digestion with Exo III, the complex is not present. The binding of Ku to the circular probe, however, is stable even in the presence of Exo III, showing that binding of Ku to the human α-MyHC promoter is independent of the presence of DNA ends.

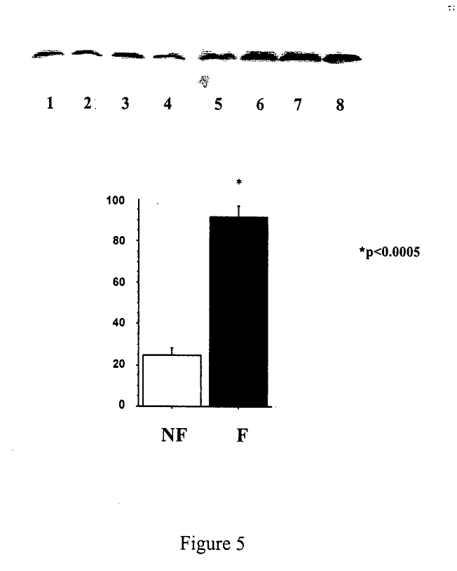

Ku70 levels are increased in human heart failure. EMSA experiments showed that Ku binding activity was increased in human failing heart extracts (see FIGS. 1A-D). To test if protein levels were also increased in human heart extracts, western blot experiments were performed using the Ku70 and Ku80 antibodies. As shown in FIG. 5, Ku70 protein levels are increased in human failing heart extracts when compared to extracts from normal hearts. The increase in the levels of Ku protein is comparable to the increase in the binding activity seen in the EMSA experiments. Thus, there is an inverse relationship between α-MyHC expression (mRNA) and Ku protein in myocardial pathologic hypertrophy and failure. Interestingly, Ku80 protein levels are not increased in human failing hearts (data not shown). This finding was surprising since in in vitro conditions up-regulation of either of these proteins requires its counterpart (personal communication). However, in pathological conditions (e.g., in response to radiation treatment (Shintani et al., 2003) the levels of Ku70 are increased but not those of Ku80.

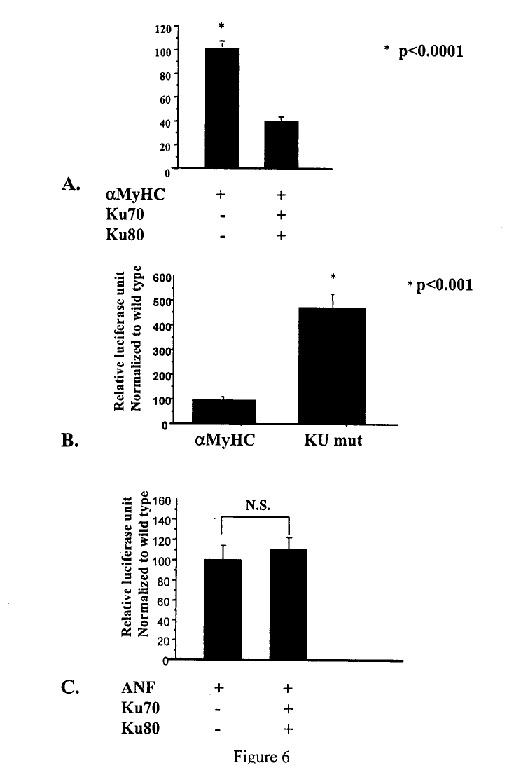

Ku represses the activity of the human α-MyHC promoter. In order to test the effect of Ku on the activity of the human α-MyHC promoter, cotransfection experiments with Ku70 and Ku80 cDNA were carried out in NRVM. As seen in FIG. 6A, expression of Ku70 and Ku80 represses the activity of the α-MyHC promoter. Transient transfection experiments were done using both Ku70 and Ku80 since overexpression of either one did not result in increased protein levels (see below). To test if the repression of the promoter was specific to the Ku binding site, an α-MyHC construct containing the two putative Ku binding sites mutated was transfected into NRVM. The mutant construct showed two-fold higher activity when compared to the wild-type (FIG. 6B) suggesting that Ku repression of the α-MyHC promoter is dependent on a direct interaction with the promoter. To test if the Ku mediated repression was specific for the α-MyHC promoter, co-transfection experiments with Ku70 and Ku80 cDNA and the atrial natriuretic factor (ANF) promoter were carried out in NRVM (FIG. 6C). ANF expression is upregulated in the hypertrophy/failure. Ku70 and Ku80 together do not repress the activity of the ANF promoter. These results suggest that Ku mediated repression in cardiac hypertrophy and failure is specific for the adult gene program and does not affect the fetal gene program, as exemplified by the ANF promoter.

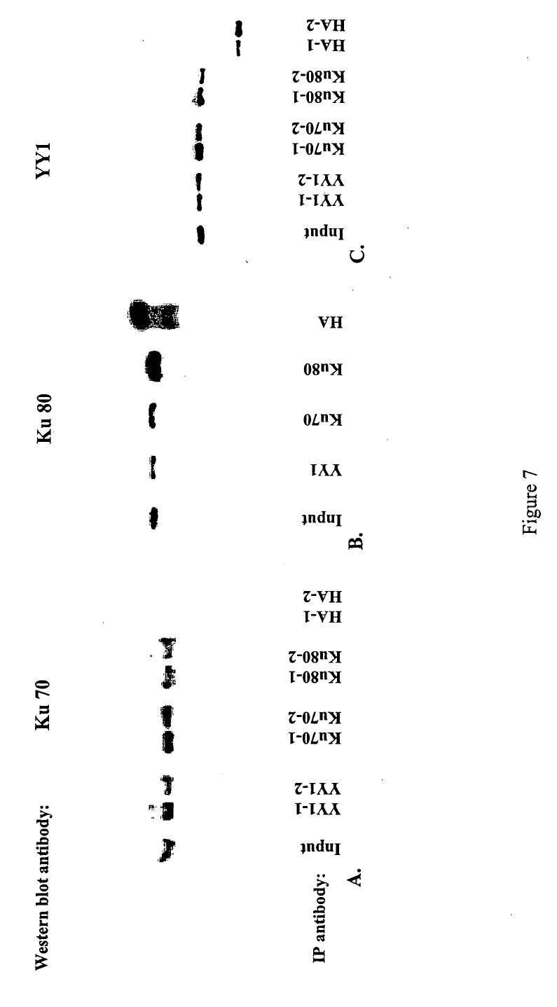

Ku70 interacts with YY1 in vitro. Ku has been shown to interact with various transcription factors so the inventors hypothesized that one or more of its interactions might facilitate the interaction of Ku with the α-MyHC promoter. Recently Ku has been shown to be important for the transcription reinitiation process (Woodard et al., 1999) and to bind directly to RNA polymerase II (Tuteja and Tuteja, 2000). It has been suggested that Ku interacts with TATA binding protein (TBP) and, most interestingly, Ku has been shown to be a promiscuous activator of transcription in yeast (Bertinato et al., 2003). In addition to its repressive effect on the αMyHC promoter, YY1 has also been shown to interact with the basic transcription machinery (Thomas and Seto, 1999) and, interestingly, recent reports have implicated YY1 as being involved in DNA repair through interaction with Poly (ADP-Ribose) (PARP) (Oei and Shi, 2001a and 2001b). Since Ku is part of the DNA repair complex and also interacts with PARP (Pleasche et al., 2000; Sartorious et al., 2000), the inventors tested the hypothesis that Ku and YY1 interact by performing co-immunoprecipitation experiments in HeLa cells. As seen in FIG. 7A and FIG. 7B, an antibody against YY1 brings down Ku70 and Ku80; in FIG. 7B and FIG. 7C, anti-Ku70 brings down YY1 and Ku80; and in FIG. 7A and FIG. 7C, anti-Ku 80 brings down Ku70 and YY1. As a control, the inventors tested if an unrelated Ab could bring down any of those proteins. As seen in FIGS. 7A, 7B and 7C, the HA Ab failed to bring down YY1, Ku70 or Ku80. All co-immunoprecipitation experiments were also done in the presence of ethidium bromide to rule out the possibility that their interaction was dependent on the presence of DNA (FIGS. 7A-C) and as shown in that figure, the presence of ethidium bromide does not change the ability of YY1 and Ku to interact showing that their interaction is independent of the presence of DNA. Although the EMSA experiments suggest that Ku can interact with the α-MyHC promoter in the absence of YY1 in vitro, interaction of YY 1 with Ku might facilitate the binding of Ku to the promoter in vivo.

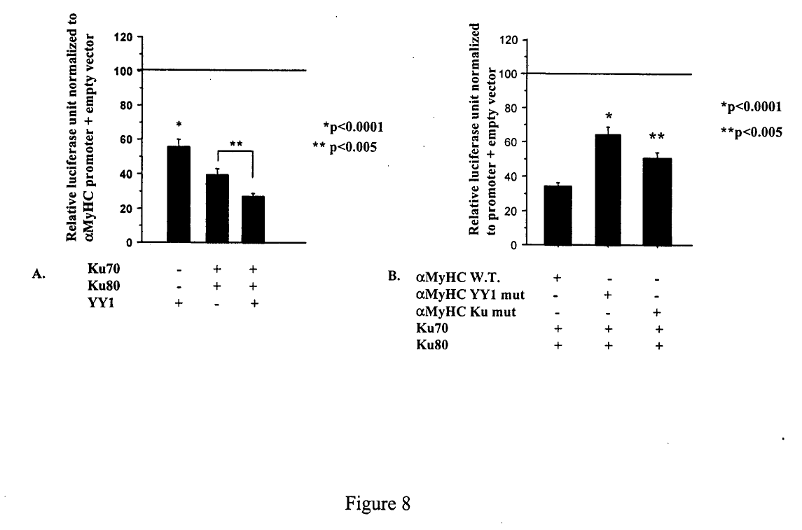

Ku70 and YY1 repress the activity of the human αMyHC promoter. Co-transfection experiments were done in NRVM using YY1, Ku70 and Ku 80 cDNAs with the α-MyHC promoter linked to luciferase. As seen in FIGS. 8A-B, the Ku complex and YY1 are capable of repressing the promoter independently. Addition of YY1 increases the repression mediated by Ku70/Ku80. The YY1 and Ku binding sites in the α-MyHC promoter are adjacent.

One possibility is that if they do not interact, they might compete for binding to the promoter. Since there is not competition in the co-transfection experiments, the inventors speculate that the interaction between the three proteins enhances their function. Moreover, co-transfection experiments using Ku70 and Ku80 and an α-MyHC promoter construct with the three YY1 sites mutated (Sucharov et al., 2003) results in a lower repression levels when compared to the wild-type promoter construct. The experiments shown in FIGS. 4A-B demonstrate that Ku70/80 can bind to the α-MyHC promoter independently of YY1, but this experiment suggests that YY1 facilitates the recruitment of the Ku complex to the promoter.

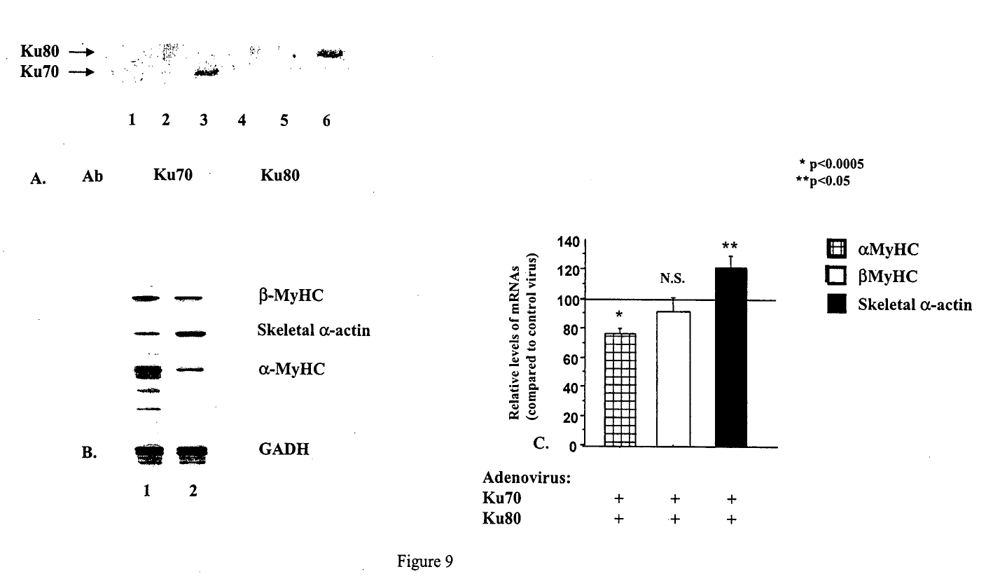

Ku70 and Ku80 repress endogenous αMyHC gene expression. In order to analyze the effect of Ku70 and Ku80 overexpression on endogenous α-MyHC gene expression, virus constructs were created that encode either Ku70 or Ku80. As mentioned above, in in vitro conditions, over expression of Ku70 is not stable in the absence of Ku80 and vice versa. As seen in FIG. 9A, over expression of either protein singly does not result in an increase in the protein levels for either protein. Over expression of Ku70 and Ku80 together however, results in increased levels of both proteins. Increased levels of Ku70 and Ku80 result in repression of endogenous α-MyHC gene expression (FIG. 9C). This repression is specific since increased levels of these proteins do not change endogenous β-MyHC gene expression levels but α-skeletal actin levels are increased. α-skeletal actin gene expression is up-regulated during cardiac hypertrophy/failure as part of the up-regulation of the fetal gene program. This data is consistent with the notion that the increase in Ku levels in human heart failure could result in repression of α-MyHC expression.

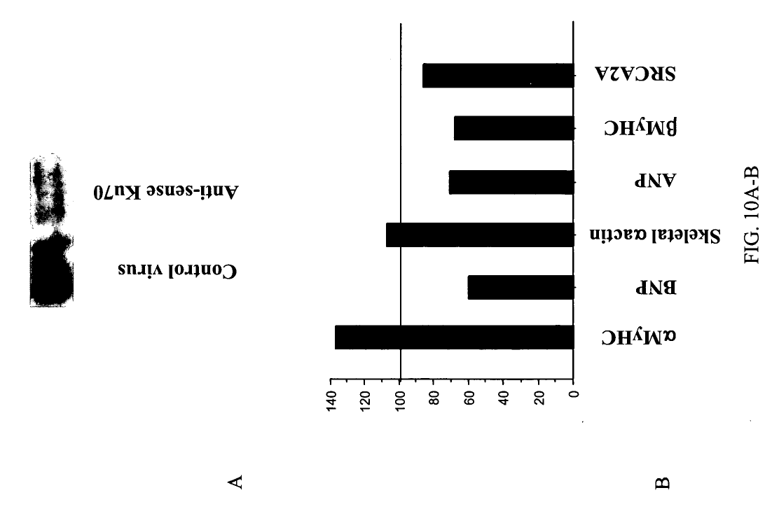

Down-regulation of Ku70 expression in cardiac myocytes results in up-regulation of α-MyHC mRNA and repression of β-MyHC mRNA. Neonate Rat Cardiac Myocytes were infected with an adenovirus construct expressing an anti-sense Ku70 cDNA construct. Expression of anti-sense Ku70 mRNA results in decreased amounts of Ku70 protein (FIG. 10A). Decrease in expression of Ku70 protein results in up-regulation of α-MyHC mRNA expression and repression of β-MyHC mRNA expression (FIG. 10B). Expression of Brain Natriuretic Peptide (BNP) and Atrial Natriuretic Peptide (ANP) are also down-regulated while expression of skeletal α-actin does not change (FIG. 10B). These results confirm the role of Ku70 in the repression of β-MyHC gene expression and its role in controlling the development of pathological cardiac hypertrophy.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods, and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.
U.S. Pat. No. 4,166,452
U.S. Pat. No. 4,196,265
U.S. Pat. No. 4,256,108
U.S. Pat. No. 4,265,874
U.S. Pat. No. 4,415,732
U.S. Pat. No. 4,458,066
U.S. Pat. No. 4,946,778
U.S. Pat. No. 5,708,158
U.S. Pat. No. 5,354,855
U.S. Pat. No. 5,440,013
U.S. Pat. No. 5,446,128
U.S. Pat. No. 5,475,085
U.S. Pat. No. 5,604,251
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,795,715
U.S. Pat. No. 5,840,833
U.S. Pat. No. 5,859,184
U.S. Pat. No. 5,888,773
U.S. Pat. No. 5,889,136
U.S. Pat. No. 5,929,237
United States App. 2002/103192
United States App. 2002/256221
United States App. 2002/61860
United States App. 2002/65282
Abraham et al., Mol Med., 8(11):750-60, 2002.
Ahmed, A., J. Am. Geriatr. Soc., 51(1):123-126, 2003.
Albert et al., Plant J, 7:649-59, 1995.
Angel et al., Mol. Cell. Biol., 7:2256, 1987.
Angel et al., Cell, 49:729, 1987b.
Angel et al., Mol. Cell. Biol., 7:2256, 1987a.
Atchison and Perry, Cell, 46:253, 1986.
Atchison and Perry, Cell, 48:121, 1987.
Atchison et al., Embo. J., 22:1347-58, 2003.
Aus. Pat. No. 6,794,700
Aus. Pat. No. 9,013,101
Aus. Pat. No. 9,013,201
Aus. Pat. No. 9,013,401
Baichwal and Sugden, Gene Transfer, Kucherlapati R, ed., New York, Plenum Press, pp. 117-148, 1986.
Banerji et al., Cell, 27(2 Pt 1):299-308, 1981.
Banerji et al., Cell, 33(3):729-740, 1983.
Barnes et al., J. Biol. Chem., 272(17):11510-11517, 1997.
Benvenisty and Neshif, Proc. Nat'l Acad. Sci. USA, 83:9551-9555, 1986.
Berkhout et al., Cell, 59:273-282, 1989.
Bertinato et al., Gene, 302:53-64, 2003.
Bhavsar et al., Genomics, 35(1):11-23, 1996.
Blanar et al., EMBO J., 8:1139, 1989.
Bliss and Lane, J. Biol. Chem., 272:5765-73, 1997.
Bodine and Ley, EMBO J., 6:2997, 1987.
Boshart et al., Cell, 41:521, 1985.
Bosher et al., Nat. Cell. Biol., 2(2):E31-E36, 2000.
Bosze et al., EMBO J., 5(7):1615-1623, 1986.
Botinelli et al., Circ. Res. 82:106-115, 1997.
Bouvagnet et al., Basic Res Cardiol., 84(1):91-102, 1989.
Braddock et al., Cell, 58:269, 1989.
Brand et al., J. Biochem. Cell. Biol., 29:1467-1470, 1997.
Bristow, Cardiology, 92:3-6, 1999.
Bulla and Siddiqui, J. Virol., 62:1437, 1986.
Camara-Clayette et al., Nucleic Acids Res., 27:1656-63, 1999.
Campbell and Villarreal, Mol. Cell. Biol., 8:1993, 1988.
Campbell, In: Monoclonal Antibody Technology, Laboratory Techniques in Biochemistry and Molecular Biology, Burden and Von Knippenberg, Eds. 13:75-83, Amsterdam, Elseview, 1984.
Campere and Tilghman, Genes and Dev., 3:537, 1989.
Campo et al., Nature, 303:77, 1983.
Caplen et al., Gene, 252:95-105, 2000.
Carniel et al., Circulation Supplement, 108:1246, 2003.
Celander and Haseltine, J. Virology, 61:269, 1987.
Celander et al., J. Virology, 62:1314, 1988.
Chandler et al., Cell, 33:489, 1983.
Chang et al., Hepatology, 14:124A, 1991.
Chang et al., Mol. Cell. Biol., 9:2153, 1989.
Chatterjee et al., Proc. Natl. Acad. Sci. USA, 86:9114, 1989.
Chen and Okayama, Mol. Cell Biol., 7:2745-2752, 1987.
Choi et al., Cell, 53:519, 1988.
Coffin, Retroviridae and Their Replication. In: Virology, Fields et al., eds., Raven Press, New York, pp. 1437-1500, 1990.
Cohen et al., J. Cell. Physiol., 5:75, 1987.
Cohen in Ph.D. Thesis (Harvard Univ., Cambridge, Mass., 1998.
Colas et al., Nature (London), 380:548-550, 1996.
Conte et al., J. Heart Lung Transplant., 17:679-685, 1998.
Cook et al., Cell, 27:487-496, 1981.
Costa et al., Mol. Cell. Biol., 8:81, 1988.
Couch et al., Am. Rev. Resp. Dis., 88:394-403, 1963.
Coupar et al., Gene, 68:1-10, 1988.
Cripe et al., EMBO J., 6:3745, 1987.
Culotta and Hamer, Mol. Cell. Biol., 9:1376, 1989.
Dandolo et al., J. Virology, 47:55-64, 1983.
De Feo et al., Ital. Heart J., 4:511-513, 2003.
De Villiers et al., Nature, 312(5991):242-246, 1984.
Deschamps et al., Science, 230:1174-1177, 1985.
DiBianco, R., Am. J. Med., 115:480-488, 2003.
Dignam et al., Nuc. Acids Res., 11:1475-89, 1983.
Dolmetsch et al., Nature, 386:855-858, 1997.
Dubensky et al., Proc. Nat'l Acad. Sci. USA, 81:7529-7533, 1984.
Dumcius et al., Medicina, 39:815-822 (2003.
Durand et al., Ann. Med., 27:311-317, 1995.
Dynan and Yoo, Nucleic Acids Res., 26:1551-9, 1998.
Edbrooke et al., Mol. Cell. Biol., 9:1908, 1989.
Edlund et al., Science, 230:912-916, 1985.
Eichhorn and Bristow, Circulation, 94:2285-2296, 1996.
Elbashir et al., EMBO, 20(23):6877-6888, 2001.
Emmel et al., Science, 246:1617-1620, 1989.
Eur. Pat. App. No. 0273085
Eur. Pat. No, 1,170,008.
Eur. Pat. No. 1,123,111.
Eur. Pat. No. 1,173,562.
Eur. Pat. No. 1,174,438.
Eur. Pat. No. 1,208,086.
Eur. Pat. No. 1,233,958.
Fabbrizio et al., Oncogene, 1999.
Fechheimer, et al., Proc Nat'l. Acad. Sci. USA 84:8463-8467, 1987.
Feng and Holland, Nature, 334:6178, 1988.
Ferkol et al., FASEB J., 7:1081-1091, 1993.
Firak and Subramanian, Mol. Cell. Biol., 6:3667, 1986.
Fire et al., Nature, 391:806-811, 1998.
Foecking and Hofstetter, Gene, 45(1):101-105, 1986.
Forster and Symons, Cell, 49:211-220, 1987.
Fraley, et al., Proc Nat'l. Acad. Sci. USA 76:3348-3352, 1979.
Franz et al., Cardoscience, 5(4):235-43, 1994.
Friedmann, Science, 244:1275-1281, 1989.
Fujita et al., Cell, 49:357, 1987.
Furumai et al., Cancer Res., 62:4916-21, 2002.
Gao et al., J. Biol. Chem., 277:25748-55, 2002.
Gefter, et al., Somatic Cell Genet., 3:231-236, 1977.
Gerlach et al., Nature, London, 328:802-805, 1987.
Geyer et al., Proc. Natl. Acad. Sci., 96:8567-8572, 1999.
Ghosh and Bachhawat, In: Liver Diseases, Targeted Diagnosis and Therapy Using Specific Receptors and Ligands. Wu et al., eds., Marcel Dekker, New York, pp. 87-104, 1991.
Ghosh-Choudhury et al., EMBO J., 6:1733-1739, 1987.
Giffin et al., Mol. Cell. Biol., 19:4065-78, 1999.
Giffin et al., Nature, 380:265-8, 1996.
Gilles et al., Cell, 33:717, 1983.
Gloss et al., EMBO J., 6:3735, 1987.
Godbout et al., Mol. Cell. Biol., 8:1169, 1988.
Goding, In: Monoclonal Antibodies: Principles and Practice, 2d ed., Academic Press, Orlando, Fla., pp. 60-61, and 71-74, 1986.
Gomez-Foix et al, J. Biol. Chem., 267:25129-25134, 1992.
Goodbourn and Maniatis, Proc. Natl. Acad. Sci. USA, 85:1447, 1988.
Goodbourn et al., Cell, 45:601, 1986.
Goodman and Gilman's “The Pharmacological Basis of Therapeutics.”
Gopal, Mol. Cell Biol., 5:1188-1190, 1985.
Gopal-Srivastava et al., J. Mol. Cell. Biol., 15(12):7081-90, 1995.
Gottlicher et al., EMBO J., 20:6969-78, 2001.
Graham and Prevec, In: Methods in Molecular Biology: Gene Transfer and Expression Protocol, E. J. Murray, ed., Humana Press, Clifton, N. J., 7:109-128, 1991.
Graham and van der Eb, Virology, 52:456-467, 1973.
Graham et al., J. Gen. Virol., 36:59-72, 1977.
Greene et al., Immunology Today, 10:272, 1989
Grishok et al., Science, 287:2494-2497, 2000.
Grosschedl and Baltimore, Cell, 41:885, 1985.
Grozinger et al., Proc. Natl. Acad. Sci. USA, 96:4868-4873, 1999.
Grunhaus and Horwitz, Seminar in Virology, 3:237-252, 1992.
Gruver et al., Endocrinology, 133:376-388, 1993.
Hajjar et al., Circulation, 86(6):1819-1826, 1992.
Han et al., Cancer Research, 60:6068-6074, 2000.
Harland and Weintraub, J. Cell Biol., 101:1094-1099, 1985.
Harlow and Lane, Antibodies: A Laboratory manual, Cold Spring Harbor Laboratory, 1988.
Haslinger and Karin, Proc. Natl. Acad. Sci. USA, 82:8572, 1985.
Hauber and Cullen, J. Virology, 62:673, 1988.
Hen et al., Nature, 321:249, 1986.
Hensel et al., Lymphokine Res., 8:347, 1989.
Hermonat and Muzycska, Proc. Nat'l Acad. Sci. USA, 81:6466-6470, 1984.
Herr and Clarke, Cell, 45:461, 1986.
Hersdorffer et al., DNA Cell Biol., 9:713-723, 1990.
Herz and Gerard, Proc. Nat'l. Acad. Sci. USA 90:2812-2816, 1993.
Hill et al., J. Biol. Chem., 277:10251-10255, 2002.
Hinnebusch et al., J. Nutr., 132:1012-7, 2002.
Hirochika et al., J. Virol., 61:2599, 1987.
Hirsch et al., Mol. Cell. Biol., 10:1959, 1990.
Ho et al, J. Biol. Chem., 270:19898-19907, 1995.
Hoey et al., Immunity, 2:461-472, 1995.
Hoffmann et al., Bioconjugate Chem., 12:51-55, 2001.
Holbrook et al., Virology, 157:211, 1987.
Hoppe-Seyler et al., J. Steroid Biochem. Mol. Biol., 78:105-111, 2004.
Horlick and Benfield, Mol. Cell. Biol., 9:2396, 1989.
Horwich, et al., J. Virol., 64:642-650, 1990.
Huang et al., Cell, 27:245, 1981.
Hug et al., Mol. Cell. Biol., 8:3065, 1988.
Hwang et al., Mol. Cell. Biol., 10:585, 1990.
Imagawa et al., Cell, 51:251, 1987.
Imbra and Karin, Nature, 323:555, 1986.
Imler et al., Mol. Cell. Biol., 7:2558, 1987.
Imperiale and Nevins, Mol. Cell. Biol., 4:875, 1984.
Jakobovits et al., Mol. Cell. Biol., 8:2555, 1988.
Jameel and Siddiqui, Mol. Cell. Biol., 6:710, 1986.
Japanese Patent Application No. 2001/348340.
Jaynes et al., Mol. Cell. Biol., 8:62, 1988.
Johnson et al., In: Biotechnology And Pharmacy, Pezzuto et al. (Eds.), Chapman and Hall, NY, 1993.
Johnson et al., Mol. Cell. Biol., 9:3393, 1989.
Jones and Shenk, Cell, 13:181-188, 1978.
Joyce, Nature, 338:217-244, 1989.
Jung et al., J. Med. Chem., 42:4669-4679, 1999.
Jung et al., Med. Chem. Lett., 7:1655-1658, 1997.
Jung, Curr. Med. Chem., 8:1505-11, 2001.
Kadesch and Berg, Mol. Cell. Biol., 6:2593, 1986.
Kaneda et al., Science, 243:375-378, 1989.
Kao et al., Genes Dev., 14:55-66, 2000.
Karin et al., Mol. Cell. Biol., 7:606, 1987.
Karin et al., Mol. Cell. Biol., 7:606, 1987.
Karlsson et al., EMBO J., 5:2377-2385, 1986.
Katinka et al., Cell, 20:393, 1980.
Kato et al., J Biol. Chem., 266(6):3361-3364, 1991.
Kawamoto et al., Mol. Cell. Biol., 8:267, 1988.
Kelly et al., J. Cell Biol., 129(2):383-96, 1995.
Ketting et al., Cell, 99(2): 133-141, 1999.
Kiledjian et al., Mol. Cell. Biol., 8:145, 1988.
Kim and Cook, Proc. Nat'l Acad. Sci. USA, 84:8788-8792, 1987.
Kim et al., J. Biol. Chem., 270:15277-84, 1995.
Kim et al., Oncogene, 18:2461-2470, 1999.
Kimura et al., Dev. Growth Differ., 39(3):257-65, 1997.
Kinugawa et al., Circ. Res., 89:591-8, 2001.
Kitazomo et al., J. Clinical Endoc. Metabol., 86(7):3430-3435, 2001.
Klamut et al., Mol. Cell. Biol., 10:193, 1990.
Klein et al., Nature, 327:70-73, 1987.
Ko and Chin, J. Biol. Chem., 278:11471-9, 2003.
Koch et al., Mol. Cell. Biol., 9:303, 1989.
Kohler and Milstein, Eur. J. Immunol., 6:511-519, 1976.
Kohler and Milstein, Nature, 256:495-497, 1975.
Koike, Journal of Radiation Research, 43:223-36, 2002.
Kolonin and Finley, Proc. Natl. Acad. Sci. USA., 95(24):14266-71, 1998.
Komastsu et al., Cancer Res., 61:4459-4466, 2001.
Kramer et al., Trends in Endoc. Metabolism, 12)7):294-300, 2001.
Kriegler and Botchan, In: Eukaryotic Viral Vectors, Gluzman (Ed.), Cold Spring Harbor: Cold Spring Harbor Laboratory, NY, 1982.
Kriegler and Botchan, Mol. Cell. Biol., 3:325, 1983.
Kriegler et al., Cell, 38:483, 1984.
Kriegler et al., Cell, 53:45, 1988.
Kudoh et al., Circ. Res., 80:139-146, 1997.
Kuhl et al., Cell, 50:1057, 1987.
Kunz et al., Nucl. Acids Res., 17:1121, 1989.
Lai and Herr, Proc. Natl. Acad. Sci. U.S.A., 89:6958-62, 1992.
Langer et al., Nuc. Acids Res., 30:3067-77, 2002.
LaPoint et al., Hypertension, 27:715-722, 1995.
LaPointe, et al., J. Biol. Chem., 263(19):9075-8, 1988.
Larsen et al., Proc Natl. Acad. Sci. USA., 83:8283, 1986.
Laspia et al., Cell, 59:283, 1989.
Latimer et al., Mol. Cell. Biol., 10:760, 1990.
Le Gal La Salle et al., Science, 259:988-990, 1993.
Lee et al., Nature, 294:228, 1981.
Lee et al., Nucleic Acids Res., 12:4191-206, 1984.
Levinson et al., Nature, 295:79, 1982.
Levrero et al., Gene, 101:195-202, 1991.
Lin et al., Genetics, 153:1245-1256, 1999.
Lin et al., J. Clin. Invest., 97:2842-2848, 1996.
Lin et al., Mol. Cell. Biol., 10:850, 1990.
Lowes et al., J. Clin. Invest., 100(9):2315-2324, 1997.
Lowes et al., N. Engl. J. Med., 346(18):1346-13467, 2002.
Luria et al., EMBO J., 6:3307, 1987.
Lusky and Botchan, Proc. Natl. Acad. Sci. USA, 83:3609, 1986.
Lusky et al., Mol. Cell. Biol., 3:1108, 1983.
Macejak and Sarnow, Nature, 353:90-94, 1991.
Mai et al., J. Med. Chem., 45:1778-1784, 2002.
Majors and Varmus, Proc. Natl. Acad. Sci. USA, 80:5866, 1983.
Mann et al., Cell, 33:153-159, 1983.
Manoria and Manoria, J. Indian. Med. Assoc., 101(5):311-312, 2003.
Marian and Roberts, Circulation, 92:1336-1347, 1995.
Markowitz et al., J. Virol., 62:1120-1124, 1988.
Massa et al., J. Med. Chem., 44:2069-2072, 2001.
Masuda et al., Mol. Cell. Biol., 15:2697-2706, 1995.
McCaffery et al., Science, 262:750-754, 1993.
McNeall et al., Gene, 76:81, 1989.
Merante et al., Mol. Cell. Biochem., 229:73-83, 2002.
Merck Index, Thirteenth Edition.
Michel and Westhof, J. Mol. Biol., 216:585-610, 1990.
Miksicek et al., Cell, 46:203, 1986.
Miyata et al., Circ. Res., 86(4):386-390, 2000.
Molkentin and Markham, J. Biol. Chem., 268:19512-20, 1993.
Montgomery et al., Proc. Nat'l. Acad. Sci., 95:15502-15507, 1998.
Mordacq and Linzer, Genes and Dev., 3:760, 1989.
Moreau et al., Nucl. Acids Res., 9:6047, 1981.
Morkin, Microsc. Res. Tech. 50:522-31, 2000.
Moss et al., J. Gen. Physiol., 108(6):473-84, 1996.
Muesing et al., Cell, 48:691, 1987.
Nakao et al., J. Clin. Invest., 100(9):2362-2370, 1997.
Ng et al., Nuc. Acids Res., 17:601, 1989.
Nicolas and Rubinstein, In: Vectors: A survey of molecular cloning vectors and their uses, Rodriguez and Denhardt, eds., Stoneham: Butterworth, pp. 494-513, 1988.
Nicolau and Sene, Biochim. Biophys. Acta, 721:185-190, 1982.
Nicolau et al., Methods Enzymol., 149:157-176, 1987.
Niimura et al., Circulation, 105(4):446-451, 2002.
Northrop et al., Nature, 369:497-502, 1994.
Oei and Shi, Biochem. Biophys. Res. Commun., 284:450-4, 2001b.
Oei and Shi, Biochem. Biophys. Res. Commun., 285:27-31, 2001a.
Olson et al, Dev. Biol., 172(1):2-14, 1995.
Ondek et al., EMBO J., 6:1017, 1987.
Ornitz et al., Mol. Cell Biol., 7:3466, 1987.
Pagani et al., Circ. Res., 63(2):380-385, 1988.
Palmiter and Solaro, Basic. Res. Cardiol., 92:63-74, 1997.
Palmiter et al., Nature, 300:611, 1982.
Park et al., J. Biol. Chem., 271:20914-20921, 1996.
Paskind et al., Virology, 67:242-248, 1975.
Patten et al., J. Biol. Chem., 271:21134-41, 1996.
PCT Application No. WO 00/44914.
PCT Application No. WO 01/14581.
PCT Application No. WO 01/18045.
PCT Application No. WO 01/36646.
PCT Application No. WO 01/38322.
PCT Application No. WO 01/42437.
PCT Application No. WO 01/68836.
PCT Application No. WO 01/70675.
PCT Application No. WO 02/26696.
PCT Application No. WO 02/26703.
PCT Application No. WO 02/30879.
PCT Application No. WO 02/46129.
PCT Application No. WO 02/46144.
PCT Application No. WO 02/50285.
PCT Application No. WO 02/51842.
PCT Application No. WO 84/03564.
PCT Application No. WO 98/33791.
PCT Application No. WO 99/32619.
Pech et al., Mol. Cell. Biol., 9:396, 1989.
Pelletier and Sonenberg, Nature, 334:320-325, 1988.
Perales, et al., Proc. Nat'l Acad. Sci. USA, 91(9):4086-4090, 1994.
Perez-Stable and Constantini, Mol. Cell. Biol., 10: 1116, 1990.
Physicians Desk Reference.
Picard and Schaffner, Nature, 307:83, 1984.
Pinkert et al., Genes and Dev., 1:268, 1987.
Pleschke et al., J. Biol. Chem., 275:40974-80, 2000.
Ponta et al., Proc. Natl. Acad. Sci. USA, 82:1020, 1985.
Porton et al., Mol. Cell. Biol., 10:1076, 1990.
Potter et al., Proc. Nat'l Acad. Sci. USA, 81:7161-7165, 1984.
Queen and Baltimore, Cell, 35:741, 1983.
Quinn et al., Mol. Cell. Biol., 9:4713, 1989.
Racher et al., Biotechnology Techniques, 9:169-174, 1995.
Ragot et al.,” Nature, 361:647-650, 1993.
Redondo et al., Science, 247:1225, 1990.
Reinhold-Hurek and Shub, Nature, 357:173-176, 1992.
Reiser et al., Am. J. Physiol. Heart. Circ. Physiol., 280(4):H1814-H1820, 2001.
Reisman and Rotter, Mol. Cell. Biol., 9:3571, 1989.
Remington's Pharmaceutical Sciences.
Renan, Radiother. Oncol., 19:197-218, 1990.
Resendez Jr. et al., Mol. Cell. Biol., 8:4579, 1988.
Rich et al., Hum. Gene Ther., 4:461-476, 1993.
Ridgeway, Mammalian Expression Vectors, In: Vectors: A Survey of Molecular Cloning Vectors and Their Uses, Rodriguez et al., eds., Stoneham: Butterworth, pp. 467-492, 1988.
Ripe et al., Mol. Cell. Biol., 9:2224, 1989.
Rippe, et al., Mol. Cell Biol., 10:689-695, 1990.
Rittling et al., Nuc. Acids Res., 17:1619, 1989.
Rooney et al., J. Heart Valve Dis., 3(1):5-9, 1994.
Rosen et al., Cell, 41:813, 1988.
Rosenfeld et al., Science, 252:431-434, 1991.
Rosenfeld, et al., Cell, 68:143-155, 1992.
Rothermel et al., Trends Cardiovasc. Med., 13:15-21, 2003.
Roux et al., Proc. Nat'l Acad. Sci. USA, 86:9079-9083, 1989.
Sadoshima and Izumo, Circ. Res., 73:424-438, 1993.
Sadoshima et al., Cell, 75:977-984, 1993)
Sakai et al., Genes and Dev., 2:1144, 1988.
Sartorius et al., J. Mol. Endocrin., 24:165-82, 2002.
Sarver et al., Science, 247:1222-1225, 1990.
Satake et al., J. Virology, 62:970, 1988.
Saunders et al., Cancer Res., 59-399-409, 1999.
Sawada et al, Nat. Cell. Biol., 5:320-9, 2003b.
Sawada et al., Nat. Cell. Biol., 5:352-7, 2003a.
Scanlon et al., Proc. Nat'l Acad. Sci. USA, 88:10591-10595, 1991.
Schaack and Guo, J. Virol., 69:3920-3, 1995.
Schaffer et al., J. Biol. Chem., 278:23141-50, 2003.
Schaffner et al., J. Mol. Biol., 201:81, 1988.
Schild-Poulter et al., J. Biol. Chem., 276:16848-56, 2001.
Schwartz et al., Circulation, 91:532-540, 1995.
Searle et al., Mol. Cell. Biol., 5:1480, 1985.
Sharp and Marciniak, Cell, 59:229, 1989.
Sharp et al., Science, 287:2431-2433, 2000.
Sharp, P. A., Genes. Dev., 13:139-141, 1999.
Shaul and Ben-Levy, EMBO J., 6:1913, 1987.
Sherman et al., Mol. Cell. Biol., 9:50, 1989.
Shintani et al., Cancer Sci., 94:894-900, 2003.
Sleigh and Lockett, J. EMBO, 4:3831, 1985.
Spalholz et al., Cell, 42:183, 1985.
Spandau and Lee, J. Virology, 62:427, 1988.
Spandidos and Wilkie, EMBO J., 2:1193, 1983.
Stemmer and Klee, Biochemistry, 33:6859-6866, 1994.
Stephens and Hentschel, Biochem. J., 248:1, 1987.
Stratford-Perricaudet and Perricaudet, In: Human Gene Transfer, O. Cohen-Haguenauer et al., eds., John Libbey Eurotext, France, pp. 51-61, 1991.
Stratford-Perricaudet et al., Hum. Gene. Ther., 1:241-256, 1990.
Stuart et al., Nature, 317:828, 1985.
Su et al., Cancer Res., 60:3137-3142, 2000.
Sucharov et al., Gene Expr., 5:93-111, 1995.
Sucharov et al., J. Biol. Chem., 278:31233-9, 2003.
Sullivan and Peterlin, Mol. Cell. Biol., 7:3315, 1987.
Swartzendruber and Lehman, J. Cell. Physiology, 85:179, 1975.
Tabara et al., Cell, 99(2):123-132, 1999.
Takahashi et al., Antibiotics, 49:453, 1996.
Takebe et al., Mol. Cell. Biol., 8:466, 1988.
Taranenko and Krause, Exp. Hematol., 28:974-84, 2000.
Taunton et al., Science, 272:371, 1996.
Tavernier et al., Nature, 301:634, 1983.
Taylor and Kingston, Mol. Cell. Biol., 10:165, 1990a.
Taylor and Kingston, Mol. Cell. Biol., 10:176, 1990b.
Taylor et al., J. Biol. Chem., 264:15160, 1989.
Temin, In: Gene Transfer, Kucherlapati R, ed., New York, Plenum Press, pp. 149-188, 1986.
Thierfelder et al., Cell, 77:701-712, 1994.
Thiesen et al., J. Virology, 62:614, 1988.
Thomas and Seto, Gene, 236:197-208, 1999.
Tong et al., Nucleic Acids Res., 30:1114-23, 2002.
Top et al., J. Infect. Dis., 124:155-160, 1971.
Treisman, Cell, 42:889, 1985.
Tronche et al., Mol. Biol. Med., 7:173, 1990.
Trudel and Constantini, Genes and Dev. 6:954, 1987.
Tur-Kaspa, et al., Mol. Cell Biol., 6:716-718, 1986.
Tuteja and Tuteja, Crit. Rev. Biochem. & Mol. Biol., 35:1-33, 2000.
Tyndell et al., Nuc. Acids. Res., 9:6231, 1981.
Usheva and Shenk, Cell, 76:1115-21, 1994.
Van den Wyngaert et al., FEBS Lett., 478:77-83, 2000.
Vannice and Levinson, J. Virology, 62:1305, 1988.
Varmus et al., Cell, 25:23-36, 1981.
Vasseur et al., Proc Natl. Acad. Sci. U.S.A., 77:1068, 1980.
Vigushin et al., Anticancer Drugs, 13:1-13, 2002.
Vigushin et al., Cancer Res., 5S, 1999.
Vigushin et al., Clinical Cancer Res., 7:971-976, 2001.
Vikstrom and Leinwand, Curr. Opin. Cell Biol., 8:97-105, 1996.
Vita et al., Biopolymers, 47:93-100, 1998.
Wagner, et al., Proc. Nat'l Acad. Sci. USA 87(9):3410-3414, 1990.
Wang and Calame, Cell, 47:241, 1986.
Waspe et al., J. Clin. Invest., 85:1206-14, 1990.
Watkins et al., Hum. Mol. Genet., 4:1721-1727, 1995.
Watkins et al., N. Engl. J. Med., 326:1108-1114, 1992.
Weber et al., Cell, 36:983, 1984.
Weinberger et al. Mol. Cell. Biol., 8:988, 1984.
Weisshoff et al. Eur. J. Biochem., 259(3):776-788, 1999.
Willis et al., J. Biol. Chem., 277:37280-91, 2002.
Wincott et al., Nucleic Acids Res., 23:2677-2684, 1995.
Winoto and Baltimore, Cell 59:649, 1989.
Wolfe et al., Nature, 385:172-176, 1997.
Wong et al., Gene, 10:87-94, 1980.
Woodard et al., J. Biol. Chem., 276:15423-33, 2001.
Workman and Kingston, Annu. Rev. Biochem., 67:545-579, 1998.
Wu and Wu, Adv. Drug Delivery Rev., 12:159-167, 1993.
Wu and Wu, Biochemistry, 27:887-892, 1988.
Wu and Wu, J. Biol. Chem., 262:4429-4432, 1987.
Xu et al., Proc. Natl. Acad. Sci., 94:12473-12478, 1997.
Yamano et al., Amer. Soc. Gene Ther., 2000.
Yamazaki et al., Circulation, 95:1260-1268, 1997.
Yang, et al., Proc Nat'l Acad. Sci. USA, 87:9568-9572, 1990.
Young et al., Handbook of Applied Therapeutics, 7.1-7.12 and 9.1-9.10, 1989.
Yutzey et al. Mol. Cell. Biol., 9:1397, 1989.
Zelenin et al., FEBS Lett., 280:94-96, 1991.
Zhou et al., Proc. Natl. Acad. Sci., 98:10572-10577, 2001.
Ziober and Kramer, J. Bio. Chem., 271(37):22915-22, 1996.

What is claimed is:

1. A method of treating cardiac hypertrophy or heart failure comprising inhibiting the function of Ku.

2. The method of claim 1, wherein inhibiting the function of Ku comprises inhibiting the interaction of Ku and YY1 or by inhibiting the interaction of Ku70 and Ku80.

3. The method of claim 1, wherein inhibiting comprises reducing the expression of Ku.

4. The method of claim 1, wherein inhibiting the function of Ku comprises inhibiting Ku's binding to the alpha myosin heavy chain promoter.

5. The method of claim 1, wherein said method further comprises inhibiting the Ku dependent repression of the alpha myosin heavy chain gene.

6. The method of claim 1, wherein inhibiting the function of Ku comprises using an agent that binds to or inactivates Ku.

7. The method of claim 3, wherein the agent that reduces the expression of Ku is an antisense construct, a ribozyme, or an siRNA.

8. The method of claim 6, wherein the agent that binds to or inactivates Ku is an antibody preparation, a Ku mimetic, a peptide, a peptide aptamer, or a small molecule.

9. The method of claim 8, wherein the antibody preparation comprises a single chain antibody.

10. The method of claim 8, wherein said antibody preparation comprises a monoclonal antibody

11. The method of claim 1, further comprising targeted delivery of the inhibitor to the heart.

12. The method of claim 11, wherein targeted delivery may be accomplished by injection of the inhibitor directly into the heart, use of an indwelling catheter or stent, use of a targeted expression vector, or a gene therapy approach.

13. The method of claim 1, further comprising administering to said patient a second therapeutic agent.

14. The method of claim 13, wherein said second therapeutic agent is selected from the group consisting of a beta blocker, an inotrope, a diuretic, ACE-I, AII antagonist, BNP, a Ca⁺⁺-channel blocker, a phosphodiesterase inhibitor, an endothelin receptor antagonist, or an HDAC inhibitor.

15. The method of claim 13, wherein said second therapy is administered at the same time as said inhibitor of Ku.

16. The method of claim 13, wherein said second therapy is administered either before or after said inhibitor of Ku.

17. The method of claim 1, wherein treating comprises improving one or more symptoms of pathologic cardiac hypertrophy.

18. The method of claim 1, wherein treating comprises improving one or more symptoms of heart failure.

19. The method of claim 17, wherein said one or more improved symptoms comprises increased exercise capacity, increased cardiac ejection volume, decreased left ventricular end diastolic pressure, decreased pulmonary capillary wedge pressure, increased cardiac output, or cardiac index, lowered pulmonary artery pressures, decreased left ventricular end systolic and diastolic dimensions, decreased left and right ventricular wall stress, decreased wall tension, increased quality of life, and decreased disease related morbidity or mortality.

20. The method of claim 18, wherein said one or more symptoms comprises progressive remodeling, ventricular dilation, decreased cardiac output, impaired pump performance, arrhythmia, fibrosis, necrosis, energy starvation, and apoptosis.

21. A method of preventing pathologic hypertrophy or heart failure comprising:
(a) identifying a patient at risk of developing pathologic cardiac hypertrophy or heart failure; and
(b) administering to said patient an inhibitor of Ku.

22. The method of claim 21, wherein said inhibitor of Ku is selected from the group consisting of a Ku siRNA molecule, a Ku antisense molecule, a Ku ribozyme molecule, a peptide, a small molecule, a Ku mimetic, a Ku aptamer, a Ku-binding single-chain antibody, or an expression construct that encodes a Ku-binding single-chain antibody.

23. The method of claim 21, wherein administering the inhibitor of Ku is performed intravenously, subcutaneously, or by direct injection into cardiac tissue.

24. The method of claim 21, wherein administering comprises oral, transdermal, sustained release, controlled release, delayed release, suppository, or sublingual administration.

25. The method of claim 21, wherein the patient at risk may exhibit one or more of a list of risk factors comprising long standing uncontrolled hypertension, uncorrected valvular disease, chronic angina, recent myocardial infarction, congenital predisposition to heart disease or pathological hypertrophy.

26. The method of claim 21, wherein the patient at risk may be diagnosed as having a genetic predisposition to cardiac hypertrophy.

26. The method of claim 21, wherein the patient at risk may have a familial history of cardiac hypertrophy.

27. A method of screening for inhibitors of cardiac hypertrophy or heart failure comprising the steps of:
(a) providing a cell having an intact alpha myosin heavy chain promoter operably linked to a reporter gene, and wherein said cell expresses sufficient levels of Ku70 and Ku80 to operably repress the α-MyHC promoter;
(b) contacting said cell with a candidate inhibitor; and
(c) monitoring said cell for an increase in expression of the reporter in the presence of said candidate inhibitor as compared to the expression of a cell in the absence of said candidate inhibitor;
wherein an increase in expression of the reporter gene in the presence of the candidate inhibitor identifies said candidate as an inhibitor of heart failure or cardiac hypertrophy.

28. The method of claim 27, wherein said cell is a cardiomyocyte.

29. The method of claim 27, wherein said cell is derived from a primary cardiomyocyte.

30. The method of claim 27, wherein contacting is performed in vitro.

31. The method of claim 27, wherein said contacting is performed in vivo.

32. The method of claim 27, wherein said candidate inhibitor is an antisense molecule.

33. The method of claim 27, wherein said candidate inhibitor is a small molecule library.

34. The method of claim 27, wherein said candidate modulator is an antibody.

35. The method of claim 27, wherein said antibody is a single chain antibody.

36. The method of claim 27, wherein said reporter protein is luciferase, β-gal, or green fluorescent protein.

37. The method of claim 27, wherein the expression level is measured using hybridization of a nucleic acid probe to a target mRNA or amplified nucleic acid product.

38. The method of claim 27, wherein expression of Ku70 and Ku80 is driven from heterologous expression constructs.

39. The method of claim 27, wherein Ku70 and Ku80 are inducibly expressed.


SEQUENCE LISTING (7 sequences)

SEQ ID NO: 1 - DNA, 12 bp, Artificial Sequence
  Description of Artificial Sequence  Synthetic Primer
gagaggggtcgg

SEQ ID NO: 2 - DNA, 1830 bp, Homo sapiens
atgtcagggtgggagtcatattacaaaaccgagggcgatgaagaagcagaggaagaacaagaagagaaccttgaagcaagtggagactataaatattcaggaagagatagtttgatttttttggttgatgcctccaaggctatgtttgaatctcagagtgaagatgagttgacaccttttgacatgagcatccagtgtatccaaagtgtgtacatcagtaagatcataagcagtgatcgagatctcttggctgtggtgttctatggtaccgagaaagacaaaaattcagtgaattttaaaaatatttacgtcttacaggagctggataatccaggtgcaaaacgaattctagagcttgaccagtttaaggggcagcagggacaaaaacgtttccaagacatgatgggccacggatctgactactcactcagtgaagtgctgtgggtctgtgccaacctctttagtgatgtccaattcaagatgagtcataagaggatcatgctgttcaccaatgaagacaacccccatggcaatgacagtgccaaagccagccgggccaggaccaaagccggtgatctccgagatacaggcatcttccttgacttgatgcacctgaagaaacctgggggctttgacatatccttgttctacagagatatcatcagcatagcagaggatgaggacctcagggttcactttgaggaatccagcaagctagaagacctgttgcggaaggttcgcgccaaggagaccaggaagcgagcactcagcaggttaaagctgaagctcaacaaagatatagtgatctctgtgggcatttataatctggtccagaaggctctcaagcctcctccaataaagctctatcgggaaacaaatgaaccagtgaaaaccaagacccggacctttaatacaagtacaggcggtttgcttctgcctagcgataccaagaggtctcagatctatgggagtcgtcagattatactggagaaagaggaaacagaagagctaaaacggtttgatgatccaggtttgatgctcatgggtttcaagccgttggtactgctgaagaaacaccattacctgaggccctccctgttcgtgtacccagaggagtcgctggtgattgggagctcaaccctgttcagtgctctgctcatcaagtgtctggagaaggaggttgcagcattgtgcagatacacaccccgcaggaacatccctccttattttgtggctttggtgccacaggaagaagagttggatgaccagaaaattcaggtgactcctccaggcttccagctggtctttttaccctttgctgatgataaaaggaagatgccctttactgaaaaaatcatggcaactccagagcaggtgggcaagatgaaggctatcgttgagaagcttcgcttcacatacagaagtgacagctttgagaaccccgtgctgcagcagcacttcaggaacctggaggccttggccttggatttgatggagccggaacaagcagtggacctgacattgcccaaggttgaagcaatgaataaaagactgggctccttggtggatgagtttaaggagcttgtttacccaccagattacaatcctgaagggaaagttaccaagagaaaacacgataatgaaggttctggaagcaaaaggcccaaggtggagtattcagaagaggagctgaagacccacatcagcaagggtacgctgggcaagttcactgtgcccatgctgaaagaggcctgccgggcttacgggctgaagagtggtctgaagaagcaggagctgctggaagccctcaccaagcacttccaggactga

SEQ ID NO: 3 - protein, 609 aa, Homo sapiens
MSGWESYYKTEGDEEAEEEQEENLEASGDYKYSGRDSLIFLVDASKAMFESQSEDELTPFDMSIQCIQSVYISKIISSDRDLLAVVFYGTEKDKNSVNFKNIYVLQELDNPGAKRILELDQFKGQQGQKRFQDMMGHGSDYSLSEVLWVCANLFSDVQFKMSHKRIMLFTNEDNPHGNDSAKASRARTKAGDLRDTGIFLDLMHLKKPGGFDISLFYRDIISIAEDEDLRVHFEESSKLEDLLRKVRAKETRKRALSRLKLKLNKDIVISVGIYNLVQKALKPPPIKLYRETNEPVKTKTRTFNTSTGGLLLPSDTKRSQIYGSRQIILEKEETEELKRFDDPGLMLMGFKPLVLLKKHHYLRPSLFVYPEESLVIGSSTLFSALLIKCLEKEVAALCRYTPRRNIPPYFVALVPQEEELDDQKIQVTPPGFQLVFLPFADDKRKMPFTEKIMATPEQVGKMKAIVEKLRFTYRSDSFENPVLQQHFRNLEALALDLMEPEQAVDLTLPKVEAMNKRLGSLVDEFKELVYPPDYNPEGKVTKRKHDNEGSGSKRPKVEYSEEELKTHISKGTLGKFTVPMLKEACRAYGLKSGLKKQELLEALTKHFQD

SEQ ID NO: 4 - DNA, 1827 bp, Mus musculus
atgtcagagtgggagtcctactacaaaactgagggcgaggaagaggaagaggaggaggagagccccgacacaggtggagaatataaatattcaggaagagatagcttgatttttctggttgatgcctccagggctatgttcgaatctcagggtgaagatgaactcacaccttttgatatgagcatccagtgtatccagagtgtgtacaccagtaagatcataagcagcgatcgggatctcctggcagtggtgttctatggaaccgagaaagataaaaattcagtgaatttcaaaaatatttatgtcttacaagatttggacaacccaggcgctaagcgagtgctagagctcgaccagtttaagggacaacaggggaagaagcacttccgagacacggttggccatgggtctgactactctttgagtgaagtgctctgggtctgtgccaacctcttcagcgacgtccagctcaagatgagtcacaagaggatcatgctgttcaccaatgaagacgacccccatggccgtgacagtgctaaagccagccgggccaggaccaaagccagcgacctccgggacactgggatcttccttgacttgatgcatctgaagaagccaggaggctttgatgtatccgtgttctacagggacatcatcaccaccgctgaggacgaggaccttggggttcacttcgaggagtcaagcaagctggaagacctgctaaggaaggttcgagccaaggagaccaaaaagcgagttctgtccaggttaaagtttaagctcggtgaagacgtagtactcatggtgggcatttataacttggtccagaaagctaacaagccttttccagtgagactctatcgggaaacaaatgaaccagtgaaaaccaagacaaggacttttaatgtaaacaccggcagtctactcctgcctagtgacaccaagcggtctctgacttacgggacacgtcagattgtgctggagaaagaggagacagaggagctgaagcggtttgatgagccaggtttgatcctcatgggctttaagcccacggtgatgctgaagaagcagcactacctgaggccctctctgttcgtgtacccagaggagtccctggtcagtgggagctcaaccttgttcagcgctctgctcaccaagtgtgtggagaagaaggtcatagcagtgtgtagatacacaccccggaagaacgtctccccgtattttgtggctttggtgccccaggaagaggagctggatgatcagaacattcaggtgactccaggaggcttccagcttgtcttcctcccttatgccgatgacaagcggaaggtgccctttactgagaaggtgacggccaaccaggagcagatagacaagatgaaggccattgttcaaaacgtccgcttcacatacaggagcgacagttttgagaatccagtcctgcagcagcacttccgcaacctggaggccctagctttggacatgatggagtcggagcaagtggtagatctgacactacccaaggttgaagccataaagaaaagactgggttccctggcagatgagtttaaagaacttgtctatcctccaggttataatcccgagggaaaagttgccaagagaaaacaagatgatgaaggttctacgagtaaaaagcccaaggtagagttatcagaagaagagctgaaggcccattttcgtaagggcacactgggtaagctcactgtacctacactgaaggacatatgcaaggctcatgggcttaagagtgggccgaagaagcaggaactgctagatgctcttatcagacacttggagaagaactga

SEQ ID NO: 5 - protein, 608 aa, Mus musculus
MSEWESYYKTEGEEEEEEEESPDTGGEYKYSGRDSLIFLVDASRAMFESQGEDELTPFDMSIQCIQSVYTSKIISSDRDLLAVVFYGTEKDKNSVNFKNIYVLQDLDNPGAKRVLELDQFKGQQGKKHFRDTVGHGSDYSLSEVLWVCANLFSDVQLKMSHKRIMLFTNEDDPHGRDSAKASRARTKASDLRDTGIFLDLMHLKKPGGFDVSVFYRDIITTAEDEDLGVHFEESSKLEDLLRKVRAKETKKRVLSRLKFKLGEDVVLMVGIYNLVQKANKPFPVRLYRETNEPVKTKTRTFNVNTGSLLLPSDTKRSLTYGTRQIVLEKEETEELKRFDEPGLILMGFKPTVMLKKQHYLRPSLFVYPEESLVSGSSTLFSALLTKCVEKKVIAVCRYTPRKNVSPYFVALVPQEEELDDQNIQVTPGGFQLVFLPYADDKRKVPFTEKVTANQEQIDKMKAIVQNVRFTYRSDSFENPVLQQHFRNLEALALDMMESEQVVDLTLPKVEAIKKRLGSLADEFKELVYPPGYNPEGKVAKRKQDDEGSTSKKPKVELSEEELKAHFRKGTLGKLTVPTLKDICKAHGLKSGPKKQELLDALIRHLEKN

SEQ ID NO: 6 - DNA, 2199 bp, Homo sapiens
atggtgcggtcggggaataaggcagctgttgtgctgtgtatggacgtgggctttaccatgagtaactccattcctggtatagaatccccatttgaacaagcaaagaaggtgataaccatgtttgtacagcgacaggtgtttgctgagaacaaggatgagattgctttagtcctgtttggtacagatggcactgacaatcccctttctggtggggatcagtatcagaacatcacagtgcacagacatctgatgctaccagattttgatttgctggaggacattgaaagcaaaatccaaccaggttctcaacaggctgacttcctggatgcactaatcgtgagcatggatgtgattcaacatgaaacaataggaaagaagtttgagaagaggcatattgaaatattcactgacctcagcagccgattcagcaaaagtcagctggatattataattcatagcttgaagaaatgtgacatctccctgcaattcttcttgcctttctcacttggcaaggaagatggaagtggggacagaggagatggcccctttcgcttaggtggccatgggccttcctttccactaaaaggaattaccgaacagcaaaaagaaggtcttgagatagtgaaaatggtgatgatatctttagaaggtgaagatgggttggatgaaatttattcattcagtgagagtctgagaaaactgtgcgtcttcaagaaaattgagaggcattccattcactggccctgccgactgaccattggctccaatttgtctataaggattgcagcctataaatcgattctacaggagagagttaaaaagacttggacagttgtggatgcaaaaaccctaaaaaaagaagatatacaaaaagaaacagtttattgcttaaatgatgatgatgaaactgaagttttaaaagaggatattattcaagggttccgctatggaagtgatatagttcctttctctaaagtggatgaggaacaaatgaaatataaatcggaggggaagtgcttctctgttttgggattttgtaaatcttctcaggttcagagaagattcttcatgggaaatcaagttctaaaggtctttgcagcaagagatgatgaggcagctgcagttgcactttcctccctgattcatgctttggatgacttagacatggtggccatagttcgatatgcttatgacaaaagagctaatcctcaagtcggcgtggcttttcctcatatcaagcataactatgagtgtttagtgtatgtgcagctgcctttcatggaagacttgcggcaatacatgttttcatccttgaaaaacagtaagaaatatgctcccaccgaggcacagttgaatgctgttgatgctttgattgactccatgagcttggcaaagaaagatgagaagacagacacccttgaagacttgtttccaaccaccaaaatcccaaatcctcgatttcagagattatttcagtgtctgctgcacagagctttacatccccgggagcctctacccccaattcagcagcatatttggaatatgctgaatcctcccgctgaggtgacaacgaaaagtcagattcctctctctaaaataaagaccctttttcctctgattgaagccaagaaaaaggatcaagtgactgctcaggaaattttccaagacaaccatgaagatggacctacagctaaaaaattaaagactgagcaagggggagcccacttcagcgtctccagtctggctgaaggcagtgtcacctctgttggaagtgtgaatcctgctgaaaacttccgtgttctagtgaaacagaagaaggccagctttgaggaagcgagtaaccagctcataaatcacatcgaacagtttttggatactaatgaaacaccgtattttatgaagagcatagactgcatccgagccttccgggaagaagccattaagttttcagaagagcagcgctttaacaacttcctgaaagcccttcaagagaaagtggaaattaaacaattaaatcatttctgggaaattgttgtccaggatggaattactctgatcaccaaagaggaagcctctggaagttctgtcacagctgaggaagccaaaaagtttctggcccccaaagacaaaccaagtggagacacagcagctgtatttgaagaaggtggtgatgtggacgatttattggacatgatatag

SEQ ID NO: 7 - protein, 732 aa, Homo sapiens
MVRSGNKAAVVLCMDVGFTMSNSIPGIESPFEQAKKVITMFVQRQVFAENKDEIALVLFGTDGTDNPLSGGDQYQNITVHRHLMLPDFDLLEDIESKIQPGSQQADFLDALIVSMDVIQHETIGKKFEKRHIEIFTDLSSRFSKSQLDIIIHSLKKCDISLQFFLPFSLGKEDGSGDRGDGPFRLGGHGPSFPLKGITEQQKEGLEIVKMVMISLEGEDGLDEIYSFSESLRKLCVFKKIERHSIHWPCRLTIGSNLSIRIAAYKSILQERVKKTWTVVDAKTLKKEDIQKETVYCLNDDDETEVLKEDIIQGFRYGSDIVPFSKVDEEQMKYKSEGKCFSVLGFCKSSQVQRRFFMGNQVLKVFAARDDEAAAVALSSLIHALDDLDMVAIVRYAYDKRANPQVGVAFPHIKHNYECLVYVQLPFMEDLRQYMFSSLKNSKKYAPTEAQLNAVDALIDSMSLAKKDEKTDTLEDLFPTTKIPNPRFQRLFQCLLHRALHPREPLPPIQQHIWNMLNPPAEVTTKSQIPLSKIKTLFPLIEAKKKDQVTAQEIFQDNHEDGPTAKKLKTEQGGAHFSVSSLAEGSVTSVGSVNPAENFRVLVKQKKASFEEASNQLINHIEQFLDTNETPYFMKSIDCIRAFREEAIKFSEEQRFNNFLKALQEKVEIKQLNHFWEIVVQDGITLITKEEASGSSVTAEEAKKFLAPKDKPSGDTAAVFEEGGDVDDLLDMI